(19) United States
(12) Patent Application Publication
(10) Pub. No.: US 2006/0281728 A1
(43) Pub. Date: Dec. 14, 2006

(54) 3-Substituted beta-lactamyl vasopressin v1a antagonists

(76) Inventors: Christophe D.G. Guillon, Bethlehem, PA (US); Ned D. Heindel, Easton, PA (US); Gary A. Koppel, Indianapolis, IN (US); Marvin J. Miller, South Bend, IN (US)

Correspondence Address: BARNES & THORNBURG LLP, 11 SOUTH MERIDIAN, INDIANAPOLIS, IN, 46204, US

(21) Appl. No.: 10/573,732

(22) PCT Filed: Oct. 1, 2004
(86) PCT No.: PCT/US04/32401

§ 371 Date: Mar. 28, 2006

Related U.S. Application Data
(60) Provisional application No. 60/508,586, filed on Oct. 3, 2003.

(51) Int. Cl. A61K 31/422; A61K 31/4178; A61K 31/4025; A61K 31/397; C07D 417/02; C07D 413/02; C07D 403/02

(52) U.S. Cl. 514/210.02; 540/200

(57) ABSTRACT

Novel 2-(azetidin-2-on-1-yl)alkanedioic acids and derivatives thereof are described. Methods for using 2-(azetidin-2-on-1-yl)alkanedioic acids and derivatives thereof in the treatment of disease states responsive to antagonism of the vasopressin V₁ₐ receptor are also described.

This invention was made in whole or in part with support from The National Institutes of Health through NIH Grant Nos. R41 HD37290 and R42 HD37290; the United States government may have certain rights in this invention.

### FIELD

The present invention relates to 2-(azetidin-2-on-1-yl)alkanedioic acids and derivatives thereof. The present invention also relates to methods of treating mammals in need of relief from disease states associated with and responsive to the antagonism of the vasopressin V₁ₐ receptor.

### BACKGROUND

Vasopressin, a neurohypophyseal neuropeptide produced in the hypothalamus, is involved in water metabolism homeostasis, renal function, mediation of cardiovascular function, non-opioid mediation of tolerance for pain, and regulation of temperature in mammals. In addition to being released into the circulatory system via the posterior pituitary, vasopressin acts as a neurotransmitter in the brain. Three vasopressin receptor subtypes, designated V₁ₐ, V_{1b}, and V₂ have been identified. The human V₁ₐ receptor has been cloned (Thibonnier et al., The Journal of Biological Chemistry, 269(5):3304-3310 (1994), the disclosure of which is incorporated herein by reference), and has been shown by radioligand binding techniques to be present in vascular smooth muscle cells, hepatocytes, blood platelets, lymphocytes and monocytes, type II pneumocytes, adrenal cortex, brain, reproductive organs, retinal epithelium, renal mesangial cells, and the A10, A7r5, 3T3, and WRK-1 cell lines (Thibonnier, Neuroendocrinology of the Concepts in Neurosurgery Series 5, (Selman, W., ed), 19-30, Williams and Wilkins, Baltimore, (1993), the disclosure of which is incorporated herein by reference).

Structural modification of vasopressin has provided a number of vasopressin agonists (Sawyer, Pharmacol. Reviews, 13:255 (1961)). In addition, several potent and selective vasopressin peptide antagonists have been designed (Lazslo et al., Pharmacological Reviews, 43:73-108 (1991); Mah and Hofbauer, Drugs of the Future, 12:1055-1070 (1987); Manning and Sawyer, Trends in Neuroscience, 7:8-9 (1984)). Finally, novel structural classes of non-peptidyl vasopressin V₁ₐ antagonists have been discovered (Yamamura et al., Science, 275:572-574 (1991); Serradiel-Le Gal et al., Journal of Clinical Investigation, 92:224-231 (1993); Serradiel-Le Gal et al., Biochemical Pharmacology, 47(4):633-641 (1994)).

Several members of the structural class of substituted 2-(azetidin-2-on-1-yl)acetic acid esters and amides have been described as synthetic intermediates for the preparation of β-lactam antibiotics. See, e.g., U.S. Pat. No. 4,751,299.

### SUMMARY

It has been found that certain coumpounds within the general class of 2-(azetidin-2-on-1-yl)alkanedioic acid derivatives elicit activity at the vasopressin V₁ₐ receptor. Described herein are novel 2-(azetidin-2-on-1-yl)alkanedioic acids, and carboxylic acid derivatives thereof, including but not limited to esters, and amides. In addition, methods useful for treating diseases and disease states that are associated with vasopressin dysfunction, and responsive to antagonism of a vasopressin receptor, such as the V₁ₐ receptor in a mammal are described. In addition, processes for preparing 2-(azetidin-2-on-1-yl)alkanedioic acids, and carboxylic acid derivatives thereof are described.

In one embodiment, compounds having the formula I are described: 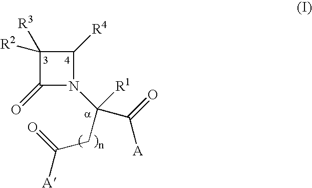
wherein:

n is an integer selected from 0, 1, and 2;

A is R⁵O—, monosubstituted amino, or disubstituted amino;

A′ is R^{5′}O—, monosubstituted amino, or disubstituted amino;

R¹ is hydrogen or C₁-C₆ alkyl;

R² is alkyl, including C₁-C₆ alkyl, alkenyl, including C₂-C₆ alkenyl, such as vinyl, alkyl, and the like, alkynyl, including C₂-C₆ alkynyl, such as ethynyl, propynyl, and the like, alkoxy, including C₁-C₄ alkoxy, alkylthio, including C₁-C₄ alkylthio, halo, haloalkyl, such as trifluoromethyl, trifluorochloroethyl, and the like, cyano, formyl, alkylcarbonyl, including C₁-C₃ alkylcarbonyl, alkoxycarbonyl, or a substituent selected from the group consisting of —CO₂R⁸, —CONR⁸R^{8′}, and —NR⁸(COR⁹);

R³ is a structure selected from the group consisting of

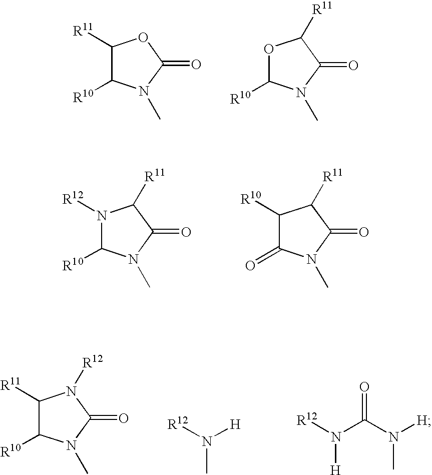

R⁴ is C₁-C₆ alkyl, C₂-C₆ alkenyl, C₂-C₆ alkynyl, C₃-C₈ cycloalkyl, C₃-C₉ cycloalkenyl, such as limonenyl, pinenyl, and the like, C₁-C₃ alkylcarbonyl, optionally substituted aryl, optionally substituted aryl(C₁-C₄ alkyl), optionally substituted aryl(C₂-C₄ alkenyl), or optionally substituted aryl(C₂-C₄ alkynyl);

R⁵ and R^{5′} are each independently selected from the group consisting of hydrogen, C₁-C₆ alkyl, C₃-C₈ cycloalkyl, (C₁-C₄ alkoxy)-(C₁-C₄ alkyl), optionally substituted aryl(C₁-C₄ alkyl), Y—, Y—(C₁-C₄ alkyl), Y′—, Y′—(C₁-C₄ alkyl), R⁶R⁷N—(C₂-C₄ alkyl), and R^{6′}R^{7′}N—(C₂-C₄ alkyl);
where Y and Y′ are each independently selected from the group consisting of tetrahydrofuryl, morpholinyl, pyrrolidinyl, piperidinyl, piperazinyl, homopiperazinyl, or quinuclidinyl; where said morpholinyl, pyrrolidinyl, piperidinyl, piperazinyl, homopiperazinyl, or quinuclidinyl is optionally N-substituted with C₁-C₄ alkyl or optionally substituted aryl(C₁-C₄ alkyl);

R⁶ is hydrogen or C₁-C₆ alkyl;

R⁷ is C₁-C₆ alkyl, C₃-C₈ cycloalkyl, optionally substituted aryl, or optionally substituted aryl(C₁-C₄ alkyl); or
R⁶ and R⁷ are taken together with the attached nitrogen atom to form an heterocycle selected from the group consisting of pyrrolidinyl, piperidinyl, morpholinyl, piperazinyl, and homopiperazinyl; where said piperazinyl or homopiperazinyl is optionally N-substitued with R¹³;

R^{6′} is hydrogen or C₁-C₆ alkyl;

R^{7′} is C₁-C₆ alkyl, C₃-C₈ cycloalkyl, optionally substituted aryl, or optionally substituted aryl(C₁-C₄ alkyl); or
R^{6′} and R^{7′} are taken together with the attached nitrogen atom to form an heterocycle selected from the group consisting of pyrrolidinyl, piperidinyl, morpholinyl, piperazinyl, and homopiperazinyl; where said piperazinyl or homopiperazinyl is optionally N-substituted with R^{13′};

R⁸ and R^{8′} are each independently selected from the group consisting of hydrogen, C₁-C₆ alkyl, C₃-C₈ cycloalkyl, optionally substituted aryl, or optionally substituted aryl(C₁-C₄ alkyl); or
R⁸ and R^{8′} are taken together with the attached nitrogen atom to form an heterocycle selected from the group consisting of optionally substituted pyrrolidinyl, piperidinyl, morpholinyl, piperazinyl, and homopiperazinyl;

R⁹ is selected from the group consisting of hydrogen, C₁-C₆ alkyl, C₃-C₈ cycloalkyl, (C₁-C₄ alkoxy)-(C₁-C₄ alkyl), optionally substituted aryl, optionally substituted aryl(C₁-C₄ alkyl), optionally substituted heteroaryl, optionally substituted heteroaryl(C₁-C₄ alkyl), and R⁸R^{8′}N—(C₁-C₄ alkyl);

R¹⁰ and R¹¹ are each independently selected from the group consisting of hydrogen, optionally substituted C₁-C₆ alkyl, optionally substituted C₃-C₈ cycloalkyl, C₁-C₄ alkoxycarbonyl, C₁-C₅ alkylcarbonyloxy, optionally substituted aryl, optionally substituted aryl(C₁-C₄ alkyl), optionally substituted aryl(C₁-C₄ alkyloxy), optionally substituted aryl(C₁-C₄ alkylcarbonyloxy), diphenylmethoxy, and triphenylmethoxy;

R¹², R¹³, and R^{13′} are each independently selected from the group consisting of hydrogen, C₁-C₆ alkyl, C₃-C₈ cycloalkyl, C₁-C₄ alkoxycarbonyl, optionally substituted aryloxycarbonyl, optionally substituted aryl(C₁-C₄ alkyl), and optionally substituted aryloyl; and

hydrates, solvates, and pharmaceutically acceptable salts thereof.

In one aspect, compounds of formula I are described, wherein A and/or A′ is a monosubstituted amino. In another aspect, compounds of formula I are described, wherein A and/or A′ is an acyclic disubstituted amino. In another aspect, compounds of formula I are described, wherein A and/or A′ is a cyclic disubstituted amino.

In another aspect, compounds of formula I are described, wherein A and/or A′ is a monosubstituted amino having the formula XNH— or X′NH—, where X and X′ are selected from the group consisting of alkyl, including C₁-C₆ alkyl, cycloalkyl, including C₃-C₈ cycloalkyl, alkoxyalkyl, including (C₁-C₄ alkoxy)-(C₁-C₄ alkyl), optionally substituted aryl, optionally substituted arylalkyl, including optionally substituted aryl(C₁-C₄ alkyl), and a group Y, Y′, Y—(C₁-C₄ alkyl), Y′—(C₁-C₄ alkyl), R⁶R⁷N—, R^{6′}R^{7′}N—, R⁶R⁷N—(C₂-C₄ alkyl), and R^{6′}R^{7′}N—(C₂-C₄ alkyl), where Y is an heterocycle.

In another aspect, compounds of formula I are described, wherein A and/or A′ is a disubstituted amino having the formula R¹⁴XN— or R^{14′}X′N—; where R¹⁴ and R^{14′} are selected from the group consisting of hydroxy, alkyl, including C₁-C₆ alkyl, alkoxycarbonyl, including C₁-C₄ alkoxycarbonyl, and benzyl; and where X and X′ are selected from the group consisting of alkyl, including C₁-C₆ alkyl, cycloalkyl, including C₃-C₈ cycloalkyl, alkoxyalkyl, including (C₁-C₄ alkoxy)-(C₁-C₄ alkyl), optionally substituted aryl, optionally substituted arylalkyl, including optionally substituted aryl(C₁-C₄ alkyl), and a group Y, Y′, Y—(C₁-C₄ alkyl), Y′—(C₁-C₄ alkyl), R⁶R⁷N—, R^{6′}R^{7′}N—, R⁶R⁷N—(C₂-C₄ alkyl), and R^{6′}R^{7′}N—(C₂-C₄ alkyl), where Y is an heterocycle.

In another aspect, compounds of formula I are described, wherein A and/or A′ is a cyclic disubstituted amino having the formula R¹⁴XN—, or R^{14′}X′N—, where R¹⁴ and X, and/or R^{14′} and X′, are taken together with the attached nitrogen atom to form an heterocycle, such as an heterocycle selected from the group consisting of pyrrolidinyl, piperidinyl, piperazinyl, and homopiperazinyl; where the heterocycle is optionally substituted with R¹⁰, R¹², R⁶R⁷N—, R^{6′}R^{7′}N—, R⁶R⁷N—(C₁-C₄ alkyl), or R^{6′}R^{7′}N—(C₁-C₄ alkyl) as defined above.

Illustrative compounds of formula I are described, wherein R¹⁴ and X, and/or R^{14′} and X′, are taken together with the attached nitrogen atom to form piperidinyl optionally substituted at the 4-position with hydroxy, alkyl, including C₁-C₆ alkyl, cycloalkyl, including C₃-C₈ cycloalkyl, alkoxy, including C₁-C₄ alkoxy, alkoxycarbonyl, including (C₁-C₄ alkoxy)carbonyl, hydroxyalkyloxyalkyl, including (hydroxy(C₂-C₄ alkyloxy))-(C₂-C₄ alkyl), R⁶R⁷N—, R⁶R⁷N-alkyl, including R⁶R⁷N—(C₁-C₄ alkyl), R^{6′}R^{7′}N—, R^{6′}R^{7′}N-alkyl, including R^{6′}R^{7′}N—(C₁-C₄ alkyl), diphenylmethyl, optionally substituted aryl, optionally substituted aryl(C₁-C₄ alkyl), or piperidin-1-yl(C₁-C₄ alkyl).

Illustrative compounds of formula I are described, wherein R¹⁴ and X and/or R^{14′} and X′ are taken together with the attached nitrogen atom to form piperazinyl optionally substituted at the 4-position with alkyl, including C₁-C₆ alkyl, cycloalkyl, including C₃-C₈ cycloalkyl, optionally substituted aryl, optionally substituted arylalkyl, including optionally substituted aryl(C₁-C₄ alkyl), α-methylbenzyl, and the like, N-alkyl acetamid-2-yl, including N—(C₁-C₅ alkyl)acetamid-2-yl, N-(cycloalkyl)acetamid-2-yl, including N—(C₃-C₈ cycloalkyl)acetamid-2-yl, R⁶R⁷N—, R^{6′}R^{7′}N—, or alkoxycarbonyl, including (C₁-C₄ alkoxy)carbonyl.

Illustrative compounds of formula I are described, wherein R¹⁴ and X and/or R^{14′} and X′ are taken together with the attached nitrogen atom to form homopiperazinyl optionally substituted in the 4-position with alkyl, including C₁-C₄ alkyl, aryl, or aryl(C₁-C₄ alkyl).

Illustrative compounds of formula I are described, wherein A and/or A′ is a disubstituted amino having the formula R¹⁴XN— or R^{14′}X′N—, where R¹⁴ and X and/or R^{14′} and X′ are taken together with the attached nitrogen atom to form an heterocycle selected from the group consisting of pyrrolidinonyl, piperidinonyl, 2-(pyrrolidin-1-ylmethyl)pyrrolidin-1-yl, 1,2,3,4-tetrahydroisoquinolin-2-yl.

In another embodiment, compounds having formula II are described: 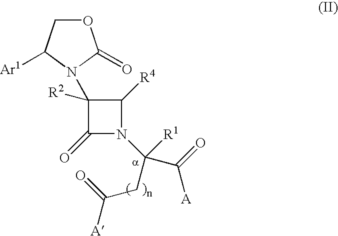
where R¹, R², R⁴, A, and A′ are as defined above, and Ar¹ is an optionally substituted aryl group.

In another embodiment, compounds having formula III are described: 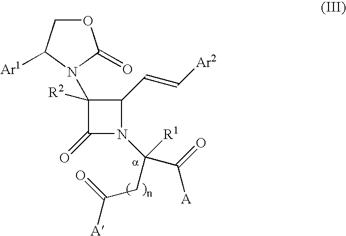
where R¹, R², A, and A′ are as defined above, and Ar¹ and Ar² are each an optionally substituted aryl group, each independently selected.

In another embodiment, a process for preparing compounds having the formulae I, II, and III is described, comprising the step of reacting a compound having the formula A 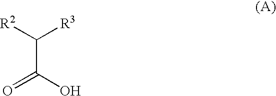
with a compound having the formula B 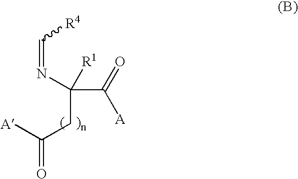
where R¹, R², R³, R⁴, A, and A′ are as defined above.

In another embodiment, a process for preparing compounds having the formula III is described, comprising the step of reacting a compound having the formula C: 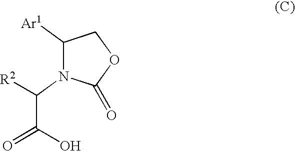
with a compound having the formula D: 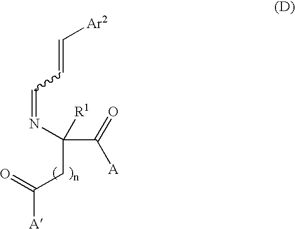
where R¹, R², R⁴, A, A′, Ar¹, and Ar² are as defined above.

In another embodiment, a method for treating a disease state responsive to the antagonism of a vasopressin V₁ₐ receptor, in a mammal in need of such treatment is described. The method comprises the step of administering to the mammal a pharmaceutically effective amount of a 2-(azetidin-2-on-1-yl)alkanedioic acid described herein, or a derivative thereof. In another embodiment, the method comprises the step of administering to the mammal a composition containing a pharmaceutically effective amount of a 2-(azetidin-2-on-1-yl)alkanedioic acid described herein, or a derivative thereof, and a pharmaceutically acceptable carrier, diluent, or excipient.

### DETAILED DESCRIPTION

In one embodiment, compounds having the formula I: 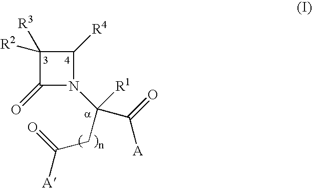
are described, wherein:

n is an integer selected from 0, 1, and 2;

A is R⁵O—, monosubstituted amino, or disubstituted amino;

A′ is R^{5′}O—, monosubstituted amino, or disubstituted amino;

R¹ is hydrogen or C₁-C₆ alkyl;

R² is alkyl, including C₁-C₆ alkyl, alkenyl, including C₂-C₆ alkenyl, such as vinyl, allyl, and the like, alkynyl, including C₂-C₆ alkynyl, such as ethynyl, propynyl, and the like, alkoxy, including C₁-C₄ alkoxy, alkylthio, including C₁-C₄ alkylthio, halo, haloalkyl, such as trifluoromethyl, trifluorochloroethyl, and the like, cyano, formyl, alkylcarbonyl, including C₁-C₃ alkylcarbonyl, alkoxycarbonyl, or a substituent selected from the group consisting of —CO₂R⁸, —CONR⁸R^{8′}, and —NR⁸(COR⁹);

R³ is a structure selected from the group consisting of

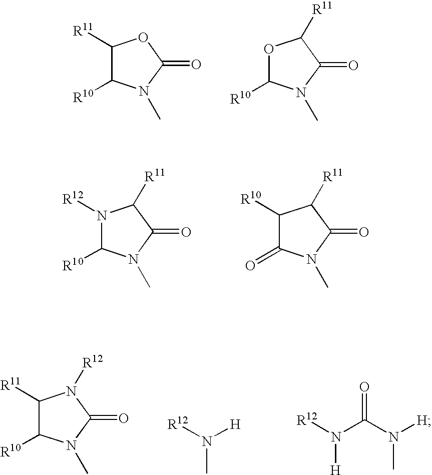

R⁴ is C₁-C₆ alkyl, C₂-C₆ alkenyl, C₂-C₆ alkynyl, C₃-C₈ cycloalkyl, C₃-C₉ cycloalkenyl, such as limonenyl, pinenyl, and the like, C₁-C₃ alkylcarbonyl, optionally substituted aryl, optionally substituted aryl(C₁-C₄ alkyl), optionally substituted aryl(C₂-C₄ alkenyl), or optionally substituted aryl(C₂-C₄ alkynyl);

R⁵ and R^{5′} are each independently selected from the group consisting of hydrogen, C₁-C₆ alkyl, C₃-C₈ cycloalkyl, (C₁-C₄ alkoxy)-(C₁-C₄ alkyl), optionally substituted aryl(C₁-C₄ alkyl), Y—, Y—(C₁-C₄ alkyl), Y′—, Y′—(C₁-C₄ alkyl), R⁶R⁷N—(C₂-C₄ alkyl), and R^{6′}R^{7′}N—(C₂-C₄ alkyl);
where Y and Y′ are each independently selected from the group consisting of tetrahydrofuryl, morpholinyl, pyrrolidinyl, piperidinyl, piperazinyl, homopiperazinyl, or quinuclidinyl; where said morpholinyl, pyrrolidinyl, piperidinyl, piperazinyl, homopiperazinyl, or quinuclidinyl is optionally N-substituted with C₁-C₄ alkyl or optionally substituted aryl(C₁-C₄ alkyl);

R⁶ is hydrogen or C₁-C₆ alkyl;

R⁷ is C₁-C₆ alkyl, C₃-C₈ cycloalkyl, optionally substituted aryl, or optionally substituted aryl(C₁-C₄ alkyl); or
R⁶ and R⁷ are taken together with the attached nitrogen atom to form an heterocycle selected from the group consisting of pyrrolidinyl, piperidinyl, morpholinyl, piperazinyl, and homopiperazinyl; where said piperazinyl or homopiperazinyl is optionally N-substitued with R¹³;

R^{6′} is hydrogen or C₁-C₆ alkyl;

R^{7′} is C₁-C₆ alkyl, C₃-C₈ cycloalkyl, optionally substituted aryl, or optionally substituted aryl(C₁-C₄ alkyl); or
R^{6′} and R^{7′} are taken together with the attached nitrogen atom to form an heterocycle selected from the group consisting of pyrrolidinyl, piperidinyl, morpholinyl, piperazinyl, and homopiperazinyl; where said piperazinyl or homopiperazinyl is optionally N-substituted with R¹³;

R⁸ and R^{8′} are each independently selected from the group consisting of hydrogen, C₁-C₆ alkyl, C₃-C₈ cycloalkyl, optionally substituted aryl, or optionally substituted aryl(C₁-C₄ alkyl); or
R⁸ and R^{8′} are taken together with the attached nitrogen atom to form an heterocycle selected from the group consisting of optionally substituted pyrrolidinyl, piperidinyl, morpholinyl, piperazinyl, and homopiperazinyl;

R⁹ is selected from the group consisting of hydrogen, C₁-C₆ alkyl, C₃-C₈ cycloalkyl, (C₁-C₄ alkoxy)-(C₁-C₄ alkyl), optionally substituted aryl, optionally substituted aryl(C₁-C₄ alkyl), optionally substituted heteroaryl, optionally substituted heteroaryl(C₁-C₄ alkyl), and R⁸R^{8′}N—(C₁-C₄ alkyl);

R¹⁰ and R¹¹ are each independently selected from the group consisting of hydrogen, optionally substituted C₁-C₆ alkyl, optionally substituted C₃-C₈ cycloalkyl C₁-C₄ alkoxycarbonyl, C₁-C₅ alkylcarbonyloxy, optionally substituted aryl, optionally substituted aryl(C₁-C₄ alkyl), optionally substituted aryl(C₁-C₄ alkyloxy), optionally substituted aryl(C₁-C₄ alkylcarbonyloxy), diphenylmethoxy, and triphenylmethoxy;

R¹², R¹³, and R^{13′} are each independently selected from the group consisting of hydrogen, C₁-C₆ alkyl, C₃-C₈ cycloalkyl, C₁-C₄ alkoxycarbonyl, optionally substituted aryloxycarbonyl, optionally substituted aryl(C₁-C₄ alkyl), and optionally substituted aryloyl; and

hydrates, solvates, and pharmaceutically acceptable salts thereof.

The general chemical terms used in the formulae described herein have their usual ordinary meanings. For example, the term “alkyl” refers to a straight-chain or optionally branched, saturated hydrocarbon, including but not limited to methyl, ethyl, n-propyl, isopropyl, n-butyl, isobutyl, sec-butyl, tert-butyl, pentyl, 2-pentyl, 3-pentyl, neopentyl, hexyl, heptyl, octyl and the like.

The term “cycloalkyl” refers to a straight-chain or optionally branched, saturated hydrocarbon, at least a portion of which forms a ring, including but not limited to cyclopropyl, cyclobutyl, cyclopentyl, methylcyclopentyl, cyclohexyl, cycloheptyl, cyclooctyl, and the like.

The term “alkenyl” includes such groups as vinyl or ethenyl, allyl or propenyl, isopropenyl, 2-butenyl, 2-methyl-2-propenyl, butadienyl, and the like.

The term “alkynyl” includes such groups as ethynyl, propynyl, 1-butynyl, hex-4-en-2-ynyl, and the like.

The term “aryl” refers to an aromatic ring or heteroaromatic ring and includes such groups as furyl, pyrrolyl, thienyl, pyridinyl, thiazolyl, oxazolyl, isoxazolyl, isothiazolyl, imidazolyl, pyrazolyl, phenyl, pyridazinyl, pyrimidinyl, pyrazinyl, thiadiazolyl, oxadiazolyl, naphthyl, indanyl, fluorenyl, quinolinyl, isoquinolinyl, benzodioxanyl, benzofuranyl, benzothienyl, and the like.

The term “optionally substituted” refers to the replacement of one or more, preferably from one to three, hydrogen atoms with one or more substitutents. Such substituents include such groups as C₁-C₄ alkyl, C₁-C₄ alkoxy, C₁-C₄ alkylthio, hydroxy, nitro, halo, carboxy, cyano, C₁-C₄ haloalkyl, C₁-C₄ haloalkoxy, amino, carbamoyl, carboxamido, amino, alkylamino, dialkylamino, alkylalkylamino, C₁-C₄ alkylsulfonylamino, and the like.

The term “heterocycle” refers to a non-aromatic cyclic structure possessing one or more heteroatoms, such as nitrogen, oxygen, sulfur, and the like, and includes such groups as tetrahydrofuryl, morpholinyl, pyrrolidinyl, piperidinyl, piperazinyl, homopiperazinyl, quinuclidinyl, and the like.

The term “alkoxy” includes such groups as methoxy, ethoxy, propoxy, isopropoxy, butoxy, tert-butoxy and the like.

The terms “acyl,” “alkanoyl,” and “aroyl” include such groups as formyl, acetyl, propanoyl, butanoyl, pentanoyl, optionally substituted benzoyl, and the like.

The term “halo” means fluoro, chloro, bromo, and iodo.

The term “alkanoyloxy” includes such groups as formyloxy, acetoxy, n-propionoxy, n-butyroxy, pivaloyloxy, and like lower alkanoyloxy groups.

The terms “optionally substituted C₁-C₄ alkyl” and “optionally substituted C₂-C₄ alkenyl” are taken to mean an alkyl or alkenyl chain which is optionally substituted with up to two methyl groups or with a C₁-C₄ alkoxycarbonyl group.

The terms “optionally substituted C₁-C₄ alkyl” and “optionally substituted C₃-C₄ cycloalkyl” are taken to mean an alkyl or cycloalkyl group which is optionally monosubstituted with a group selected from hydroxy, protected hydroxy, alkyl, protected carboxyl, carbamoyl, benzylthio, and alkylthio.

The term “(C₁-C₄ alkyl)” as used in for example “aryl(C₁-C₄ alkyl)”, “(C₁-C₄ alkoxy)-(C₁-C₄ alkyl)”, and the like, refers to a saturated linear or branched divalent alkyl chain of from one to four carbons bearing for example aryl, C₁-C₄ alkoxy, and the like, as a substituent and includes such groups as for example benzyl, phenethyl, phenpropyl, α-methylbenzyl, methoxymethyl, ethoxyethyl, and the like.

The term “optionally substituted phenyl” is taken to mean a phenyl radical optionally substituted with one, two, or three substituents each independently selected from the group consisting of C₁-C₄ alkyl, C₁-C₄ alkoxy, hydroxy, halo, nitro, trifluoromethyl, sulfonamido, cyano, carbamoyl, amino, mono(C₁-C₄ alkyl)amino, di(C₁-C₄ alkyl)amino, C₁-C₄ alkylsulfonylamino, and indol-2-yl.

The term “protected amino” refers to amine protecting groups used to protect the nitrogen of the β-lactam ring during preparation or subsequent reactions. Examples of such groups are benzyl, 4-methoxybenzyl, 4-methoxyphenyl, or trialkylsilyl, for example trimethylsilyl.

The term “protected carboxy” refers to the carboxy group protected or blocked by a conventional protecting group commonly used for the temporary blocking of the acidic carboxy. Examples of such groups include lower alkyl, for example tert-butyl, halo-substituted lower alkyl, for example 2-iodoethyl and 2,2,2-trichloroethyl, benzyl and substituted benzyl, for example 4-methoxybenzyl and 4-nitrobenzyl, diphenylmethyl, alkenyl, for example allyl, trialkylsilyl, for example trimethylsilyl and tert-butyldiethylsilyl and like carboxy-protecting groups.

The term “antagonist”, as it is used in the description of this invention, is taken to mean a full or partial antagonist. A compound which is a partial antagonist at the vasopressin V₁ₐ receptor must exhibit sufficient antagonist activity to inhibit the effects of vasopressin or a vasopressin agonist at an acceptable dose. While a partial antagonist of any intrinsic activity may be useful, the partial antagonists illustratively show at least about 50% antagonist effect, or at least about 80% antagonist effect. The term also includes compounds that are full antagonists of the vasopressin V₁ₐ receptor.

The compounds of the present invention possess an azetidinone core structure that includes asymmetric carbon atoms at C(3) and C(4), creating four stereoisomeric configurations, as illustrated by the following: 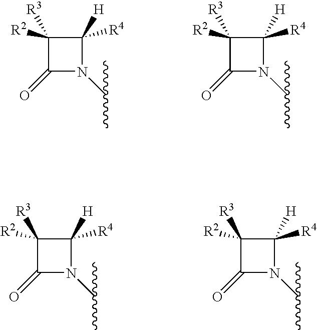
The compounds described herein may, therefore, exist as single diastereomers, as a racemic mixture, or as a mixture of various diastereomers. It is understood that in some applications, certain stereoisomers or mixtures of stereoisomers may be used, while in others applications, other stereoisomers or mixtures of stereoisomers may be used. In some embodiments, a single stereoisomer is described, such as the azetidinone core structure having the (3S,4R)-diastereomeric configuration.

It is also understood, that except when A=A′ and n=0, the α-carbon bearing R¹ is also chiral. Furthermore, the groups selected for R¹, R², R³, R⁴, A, and A′ may also include chiral centers. For example, when R³ is 4-substituted oxazolidin-2-on-3-yl, the 4-position of that ring is asymmetric. In addition, when R³ is 2,5-substituted oxazolidin-4-on-3-yl or 1,2,5-trisubstituted imidazolidin-4-on-3-yl, the 2- and 5-carbons of those rings are each asymmetric. Finally, when R³ is succinimido and one of R¹⁴ and R¹⁵ is hydrogen, the carbon bearing the non-hydrogen substituent is also asymmetric. Therefore, additional stereoisomers are collectively represented by formula I. While compounds possessing all combinations of stereochemical purity are contemplated by the present description, it is appreciated that in many cases at least one of these chiral centers described above may be present as a single absolute configuration in a compound described herein. In one illustrative aspect, the compounds described herein have the (αR,3S,4R) absolute configuration or the (αS,3S,4R) absolute configuration.

Illustrative embodiments of the compounds described herein include compounds having the formula I where:

(aa) A is R⁵O—;

(ab) A is R⁵O—, and R⁵ is C₁-C₆ alkyl;

(ac) A is R⁵O—, and R⁵ is optionally substituted aryl(C₁-C₄ alkyl);

(ad) A is a monosubstituted amino of the formula XNH—;

(ae) A is a disubstituted amino having the formula R¹⁴XN—;

(af) A′ is a monosubstituted amino having the formula X′NH—;

(ag) A′ is a disubstituted amino having the formula R^{14′}X′N—;

(ah) A′ is R^{5′}O—;

(ai) A′ is R^{5′}O—, and R^{5′} is C₁-C₆ alkyl;

(aj) A′ is R^{5′}O—, and R^{5′} is optionally substituted aryl(C₁-C₄ alkyl);

(ak) A is XNH— or R¹⁴XN, and X is optionally substituted aryl(C₁-C₄ alkyl);

(al) A is XNH— or R¹⁴XN, and X is R⁶R⁷N—(C₁-C₄ alkyl);

(am) A is XNH— or R¹⁴XN, X is R⁶R⁷N—(C₁-C₄ alkyl), and R⁶ and R⁷ are taken together with the attached nitrogen atom to form an heterocycle;

(an) A is R¹⁴XN, and R¹⁴ and X are taken together with the attached nitrogen atom to form an heterocycle;

(ao) A is R¹⁴XN, R¹⁴ and X are taken together with the attached nitrogen atom to form an heterocycle, and the heterocycle is optionally substituted with an optionally substituted aryl(C₁-C₄ alkyl), an heterocycle Y, or C₃-C₈ cycloalkyl;

(ap) R¹ is hydrogen;

(aq) R¹ is C₁-C₆ alkyl;

(ar) R¹ is C₁-C₂ alkyl;

(as) R³ is 4-substituted oxazolidin-2-on-3-yl;

(at) R³ is 4,5-disubstituted oxazolidin-2-on-3-yl;

(au) R³ is 2-substituted oxazolidin-4-on-3-yl;

(av) R³ is 2-substituted imidazolidin-4-on-3-yl;

(aw) R³ is 1,2-disubstituted imidazolidin-4-on-3-yl;

(ax) R³ is 5-substituted imidazolidin-2-on-1-yl;

(ay) R³ is 4,5-disubstituted imidazolidin-4-on-1-yl;

(az) R⁴ is optionally substituted 2-aryleth-1-yl;

(ba) R⁴ is optionally substituted 2-arylethen-1-yl;

(bb) A′ is R^{14′}X′N—, and R^{14′} is benzyl;

(bc) A′ is X′NH— or R^{14′}X′N—, and X′ is an heterocycle Y;

(bd) A is XNH— or R¹⁴XN—, and X is optionally substituted aryl(C₁-C₄ alkyl);

(be) A is XNH— or R¹⁴XN—, X is optionally substituted aryl(C₁-C₄ alkyl), and aryl is optionally substituted phenyl;

(bf) A′ is X′NH— or R^{14′}XN—, and X′ is R^{6′}R^{7′}N—(C₁-C₄ alkyl);

(bg) A′ is X′NH— or R^{14′}XN—, and X′ is R^{6′}R^{7′}N—;

(bh) A′ is X′NH— or R^{14′}X′N—, X′ is R^{6′}R^{7′}N—, and R^{6′} is C₁-C₆ alkyl;

(bi) A′ is X′NH— or R^{14′}X′N—, X′ is R^{6′}R^{7′}N—, and R^{7′} is C₁-C₆ alkyl;

(bj) A′ is X′NH— or R^{1′}X′N—, X′ is R^{6′}R^{7′}N—, and R^{6′} and R^{7′} are taken together with the attached nitrogen atom to form an heterocycle;

(bk) A is XNH— or R¹⁴X—, X is R⁶R⁷N—, and R⁶ and R⁷ are the same and are C₁-C₆ alkyl;

(bl) A′ is X′NH— or R^{14′}X′N, X′ is R^{6′}R^{7′}N—, and R^{14′} and X′ taken together with the nitrogen to which they are attached form pyrrolidinyl, piperidinyl, piperazinyl; where said pyrrolidinyl, piperidinyl, or piperazinyl is optionally substituted with an heterocycle Y′ or with R⁶R⁷N—(C₁-C₄ alkyl);

(bm) A′ is X′NH— or R^{14′}X′N—, X′ is R^{6′}R^{7′}N—, and R^{14′} and X′ taken together with the nitrogen to which they are attached form piperidinyl optionally substituted at the 4-position with hydroxy, C₁-C₆ alkyl, C₃-C₈ cycloalkyl, C₁-C₄ alkoxy, (C₁-C₄ alkoxy)carbonyl, (hydroxy(C₁-C₄ alkyloxy))-(C₁-C₄ alkyl), R⁶R⁷N—, R⁶R⁷N—C₁-C₄ alkyl), phenyl, phenyl(C₁-C₄ alkyl), optionally substituted phenyl(C₁-C₄ alkyl), furyl(C₁-C₄ alkyl), pyridinyl(C₁-C₄ alkyl), thienyl(C₁-C₄ alkyl), or piperidin-1-yl(C₁-C₄ alkyl);

(bn) A′ is X′NH— or R^{14′}X′N—, X′ is R^{6′}R^{7′}N—, and R^{14′} and X′ taken together with the nitrogen to which they are attached form piperazinyl optionally substituted at the 4-position with C₁-C₆ alkyl, C₃-C₈ cycloalkyl, optionally substituted phenyl, optionally substituted phenyl(C₁-C₄ alkyl), N—(C₁-C₅ alkyl)acetamid-2-yl, N—(C₃-C₈ cycloalkyl)acetamid-2-yl, R⁶R⁷N—, or (C₁-C₄ alkoxy)carbonyl; and

(bo) A′ is X′NH— or R^{14′}X′N—, X′ is R^{6′}R^{7′}N—, and R^{14′} and X′ taken together with the nitrogen to which they are attached form homopiperazinyl optionally substituted in the 4-position with C₁-C₄ alkyl, phenyl, or phenyl(C₁-C₄ alkyl).

It is appreciated that the classes of compounds described above may be combined to form additional illustrative classes. An example of such a combination of classes may be a class of compounds wherein A is a monosubstituted amino having the formula XNH—, where X is optionally substitued aryl(C₁-C₄ alkyl), and A′ is a disubstituted amino having the formula R^{14′}X′N—, where R^{14′} and X′ are taken together with the attached nitrogen atom to form an heterocycle, such as piperidine, peperazine, and the like. Further combinations of the classes of compounds described above are contemplated in the present invention.

In another embodiment, compounds having the following formula: 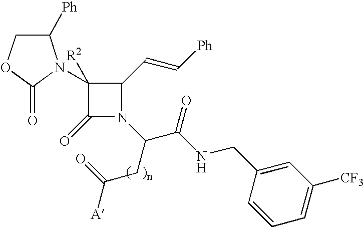
are described, where n and A′ are as described above; R² is illustratively C₁-C₆ alkyl, such as methyl, ethyl, propyl, and the like; haloalkyl, such as trifluoromethyl, chlorodifluoromethyl, tetrafluoroethyl, and the like; C₁-C₄ alkoxy, such as methoxy, ethoxy, and the like; haloalkoxy, such as trifluoromethoxy, chlorodifluoromethoxy, tetrafluoroethoxy, and the like; C₁-C₄ alkylthio, such as methylthio, ethylthio, and the like; cyano; formyl; and halo, such as fluoro and chloro.

It is understood that the above formula represents 16 different stereoisomeric configurations. It is appreciated that certain stereoisomers may be more biologically active than others. Therefore, the above formula contemplates herein all possible stereoisomers, as well as various mixtures of each stereoisomer. Illustratively, the following pair of enantiomers: 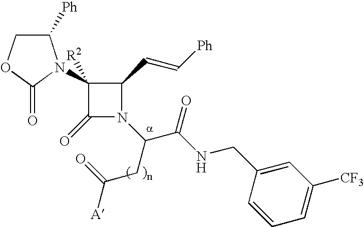
is described, where the sterochemistry at the “α” carbon is either (R) or (S).

In one aspect, the group A′ includes, but is not limited to 2-(piperidin-1-yl)ethylamino, 4-(piperidin-1-yl)piperidin-1-yl, 4-(phenylethyl)piperazin-1-yl, fur-2-ylmethylamino, 4-(pyrrolidin-1-yl)piperazin-1-yl, 4-(3-trifluoromethylphenyl)piperazin-1-yl, 4-(benzyloxycarbonyl)piperazin-1-yl, 4-[2-(2-hydroxyethoxy)ethyl]piperazin-1-yl, 4-benzylpiperazin-1-yl, 4-(3,4-methylenedioxybenzyl)piperazin-1-yl, 4-phenylpiperazin-1-yl, 4-(3-phenylprop-2-enyl)piperazin-1-yl, 4-ethylpiperazin-1-yl, 2-(dimethylamino)ethylamino, 4-(pyrrolidin-1-ylcarbonylmethyl)piperazin-1-yl, 4-(1-methylpiperidin-4-yl)piperazin-1-yl, 4-butylpiperazin-1-yl, 4-isopropylpiperazin-1-yl, 4-pyridylmethylamino, 3-(dimethylamino)propylamino, 1-benzylpiperidin-4-ylamino, N-benzyl-2-(dimethylamino)ethylamino, 3-pyridylmethylamino, 4-(cyclohexyl)piperazin-1-yl, 4-(2-cyclohexylethyl)piperazin-1-yl, 4-[2-(morpholin-4-yl)ethyl]piperazin-1-yl, 4-(4-tert-butylbenzyl)piperazin-1-yl, 4-[2-(piperidin-1-yl)ethyl]piperazin-1-yl, 4-[3-(piperidin-1-yl)propyl]piperazin-1-yl, 4-[2-(N,N-dipropylamino)ethyl]piperazin-1-yl, 4-[3-(N,N-diethylamino)propyl]piperazin-1-yl, 4-[2-(dimethylamino)ethyl]piperazin-1-yl, 4-[3-(pyrrolidin-1-yl)propyl]piperazin-1-yl, 4-(cyclohexylmethyl)piperazin-1-yl, 4-cyclopentylpiperazin-1-yl, 4-[2-(pyrrolidin-1-yl)ethyl]piperazin-1-yl, 4-[2-(thien-2-yl)ethyl]piperazin-1-yl, 4-(3-phenylpropyl)piperazin-1-yl, 4-[2-(N,N-diethylamino)ethyl]piperazin-1-yl, 4-benzylhomopiperazin-1-yl, 4-(bisphenylmethyl)piperazin-1-yl, 3-(4-methylpiperazin-1-yl)propylamino, (+)-3(S)-1-benzylpyrrolidin-3-ylamino, 2-pyridylmethylamino, and 4-[2-(piperidin-1-yl)ethyl]piperidin-1-yl.

In another aspect, the integer n is 1 or 2, and the group A′ includes, but is not limited to 2-(piperidin-1-yl)ethylamino, 4-(piperidin-1-yl)piperidin-1-yl, 2-(pyrid-2-yl)ethylamino, morpholin-4-ylamino, 4-(pyrrolidin-1-yl)piperazin-1-yl, 4-(3-trifluorophenyl)piperazin-1-yl, 4-(benzyloxycarbonyl)piperazin-1-yl, 4-[2-(2-hydroxylethoxy)ethyl]piperazin-1-yl, 4-benzylpiperazin-1-yl, 4-(3,4-methylenedioxybenzyl)piperazin-1-yl, 4-phenylpiperazin-1-yl, 4-(3-phenylprop-2-enyl)piperazin-1-yl, 4-ethylpiperazin-1-yl, 2-(dimethylamino)ethylamino, 4(pyrrolidin-1-ylcarbonylmethyl)piperazin-1-yl, 4-(1-methylpiperidin-4-yl)piperazin-1-yl, 4-butylpiperazin-1-yl, 4-isopropylpiperazin-1-yl, 4-pyridylmethylamino, 3-(dimethylamino)propylamino, 1-benzylpiperidin-4-ylamino, N-benzyl-2-(dimethylamino)ethylamino, 3-pyridylmethylamino, 4-cyclohexylpiperazin-1-yl, 4-(2-cyclohexylethyl)piperazin-1-yl, 4-[2-(morpholin-4-yl)ethyl]piperazin-1-yl, 4-(4-tert-butylbenzyl)piperazin-1-yl, 4-[2-(piperidin-1-yl)ethyl]piperazin-1-yl, 4-[3-(piperidin-1-yl)propyl]piperazin-1-yl, 4-[2-(diisopropylamino)ethyl]piperazin-1-yl, 4-[3-(diethylamino)propyl]piperazin-1-yl, 4-(2-dimethylaminoethyl)piperazin-1-yl, 4-[3-(pyrrolidin-1-yl)propyl]piperazin-1-yl, 4-(cyclohexylmethyl)piperazin-1-yl, 4-[2-(piperidin-1-yl)ethyl]piperidin-1-yl, 4-propyl-piperazin-1-yl, 4-[N-(isopropyl)acetamid-2-yl]piperazin-1-yl, and 3-benzyl-hexahydro-(1H)-1,3-diazepin-1-yl.

In another embodiment, compounds having the following formula: 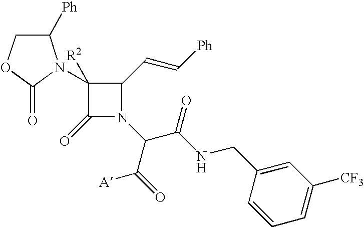
are described, where A′ is as described above; R² is illustratively C₁-C₆ alkyl, such as methyl, ethyl, propyl, and the like; haloalkyl, such as trifluoromethyl, chlorodifluoromethyl, tetrafluoroethyl, and the like; C₁-C₄ alkoxy, such as methoxy, ethoxy, and the like; haloalkoxy, such as trifluoromethoxy, chlorodifluoromethoxy, tetrafluoroethoxy, and the like; C₁-C₄ alkylthio, such as methylthio, ethylthio, and the like; cyano; formyl; and halo, such as fluoro and chloro.

It is understood that the above formula represents 16 different stereoisomeric configurations. It is appreciated that certain stereoisomers may be more biologically active than others. Therefore, the above formula contemplates herein all possible stereoisomers, as well as various mixtures of each stereoisomer. Illustratively, the following pair of enantiomers: 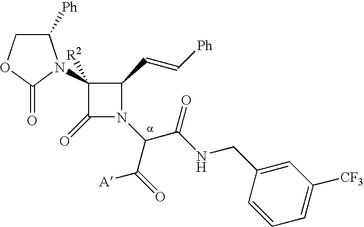
is described, where the sterochemistry at the “α” carbon is either (R) or (S).

In one aspect, the group A′ includes, but is not limited to 2-(piperidin-1-yl)alkylamino, 4-(piperidin-1-yl)piperidin-1-yl, 4-(2-arylalkyl)piperazin-1-yl, 1-arylalkylpiperidin-4-ylamino, 4-alkylpiperazin-1-yl, such as 4-butyl, 4-isopropyl, 4-cyclohexylpiperazin-1-yl, and the like, and 4-[2-(piperidin-1-yl)ethyl]piperidin-1-yl.

In another embodiment, compounds having the formula: 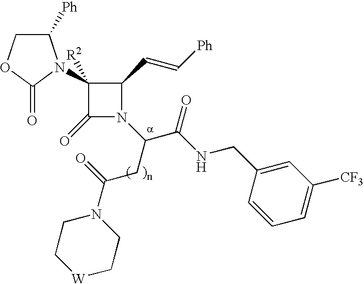
are described, where n is as described above; R² is illustratively C₁-C₆ alkyl, such as methyl, ethyl, propyl, and the like; haloalkyl such as trifluoromethyl chlorodifluoromethyl, tetrafluoroethyl, and the like; C₁-C₄ alkoxy, such as methoxy, ethoxy, and the like; haloalkoxy, such as trifluoromethoxy, chlorodifluoromethoxy, tetrafluoroethoxy, and the like; C₁-C₄ alkylthio, such as methylthio, ethylthio, and the like; cyano; formyl; and halo, such as fluoro and chloro; W is either carbon or ntrogen, each optionally substituted with a carbocyclyl substituent, such as cyclopentyl, cyclohexyl, and the like, or an an heterocyclyl substituent, such as pyrrolidinyl, piperidinyl, and the like; and the sterochemistry at the “α” carbon is either (R) or (S). The following compounds:

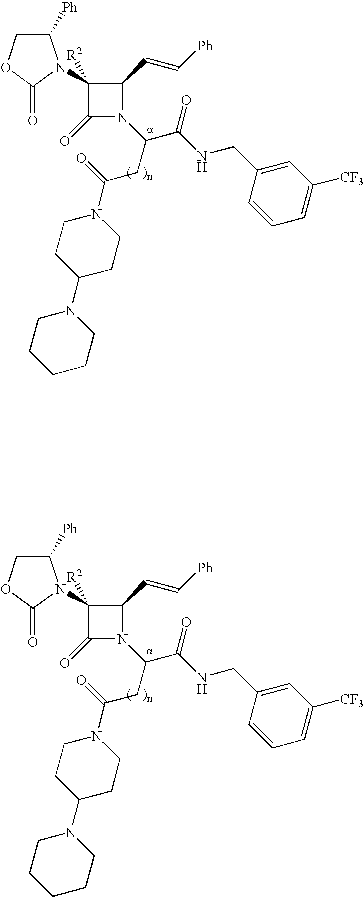

are illustrative of this embodiment. In one aspect, when n is 1, W as defined above is a carbon atom substituted with piperidin-1-yl. In another aspect, when n is 2, W as defined above is a nitrogen atom susbstituted with cyclohexyl. The following compounds are illustrative of this embodiment:

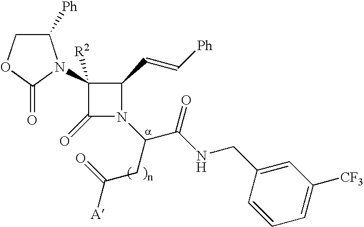

| R² | α-configuration | n | A′ |
|---|---|---|---|
| Me | R | 1 | 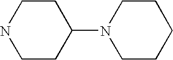 |
| Me | S | 2 | 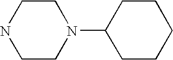 |
| MeS | R | 1 | 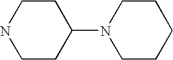 |
| MeS | S | 2 | 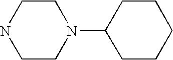 |
| MeO | R | 1 | 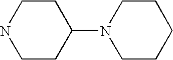 |
| MeO | S | 2 | 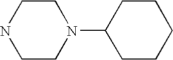 |
| CN | R | 1 | 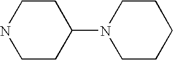 |
| CN | S | 2 | 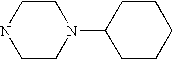 |
| F | R | 1 | 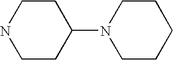 |
| F | S | 2 | 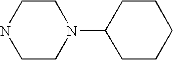 |
| CHO | R | 1 | 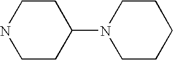 |
| CHO | S | 2 | 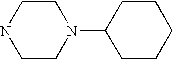 |

In another embodiment, compounds having the following formula: 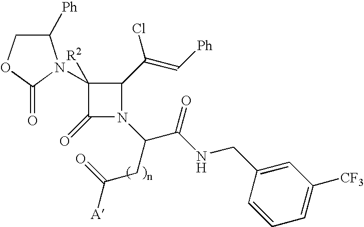
are described, where n and A′ are as described above; R² is illustratively C₁-C₆ alkyl, such as methyl, ethyl, propyl, and the like; haloalkyl, such as trifluoromethyl, chlorodifluoromethyl, tetrafluoroethyl, and the like; C₁-C₄ alkoxy, such as methoxy, ethoxy, and the like; haloalkoxy, such as trifluoromethoxy, chlorodifluoromethoxy, tetrafluoroethoxy, and the like; C₁-C₄ alkylthio, such as methylthio, ethylthio, and the like; cyano; formyl; and halo, such as fluoro and chloro.

It is understood that the above formula represents 16 different stereoisomeric configurations. It is appreciated that certain stereoisomers may be more biologically active than others. Therefore, the above formula contemplates herein all possible stereoisomers, as well as various mixtures of each stereoisomer. Illustratively, the following pair of enantiomers: 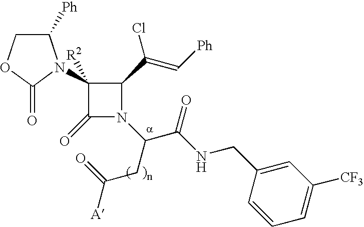
is described, where the sterochemistry at the “α” carbon is either (R) or (S).

In one aspect, the group A′ includes, but is not limited to 4-cyclohexylpiperazin-1-yl, 4-(pyrrolidin-1-yl)piperazin-1-yl, 4-ethylpiperazin-1-yl, 4-n-butylpiperazin-1-yl, and 4-isopropylpiperazin-1-yl.

In another embodiment, compounds having the following formula: 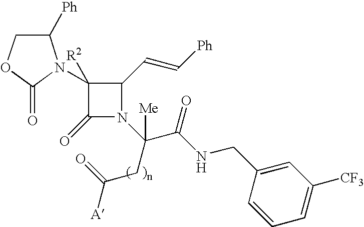
are described, where n and A′ are as described above; R² is illustratively C₁-C₆ alkyl, such as methyl, ethyl, propyl, and the like; haloalkyl, such as trifluoromethyl, chlorodifluoromethyl, tetrafluoroethyl, and the like; C₁-C₄ alkoxy, such as methoxy, ethoxy, and the like; haloalkoxy, such as trifluoromethoxy, chlorodifluoromethoxy, tetrafluoroethoxy, and the like; C₁-C₄ alkylthio, such as methylthio, ethylthio, and the like; cyano; formyl; and halo, such as fluoro and chloro.

It is understood that the above formula represents 16 different stereoisomeric configurations. It is appreciated that certain stereoisomers may be more biologically active than others. Therefore, the above formula contemplates herein all possible stereoisomers, as well as various mixtures of each stereoisomer. Illustratively, the following pair of enantiomers: 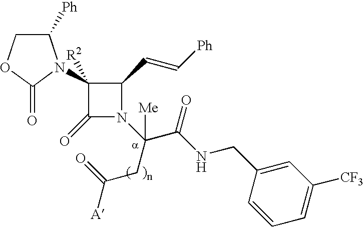
is described, where the sterochemistry at the “α” carbon is either (R) or (S).

In one aspect, the group A′ includes, but is not limited to optionally substituted 4-piperidin-1-ylpiperidinyl, optionally substituted 4-arylalkylpiperazinyl, and optionally substituted 4-cycloalkylpiperazinyl.

In another embodiment, compounds having the following formula: 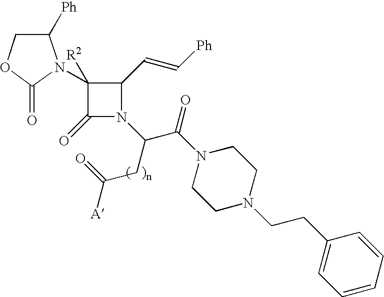
are described, where n and A′ are as described above; R² is illustratively C₁-C₆ alkyl, such as methyl, ethyl, propyl, and the like; haloalkyl, such as trifluoromethyl, chlorodifluoromethyl, tetrafluoroethyl, and the like; C₁-C₄ alkoxy, such as methoxy, ethoxy, and the like; haloalkoxy, such as trifluoromethoxy, chlorodifluoromethoxy, tetrafluoroethoxy, and the like; C₁-C₄ alkylthio, such as methylthio, ethylthio, and the like; cyano; formyl; and halo, such as fluoro and chloro.

It is understood that the above formula represents 16 different stereoisomeric configurations. It is appreciated that certain stereoisomers may be more biologically active than others. Therefore, the above formula contemplates herein all possible stereoisomers, as well as various mixtures of each stereoisomer. Illustratively, the following pair of enantiomers: 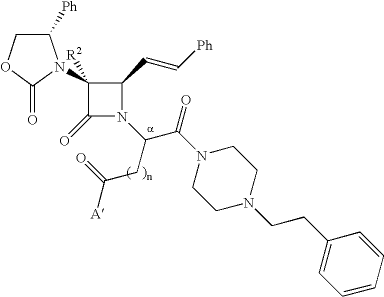
is described, where the sterochemistry at the “α” carbon is either (R) or (S).

In one aspect, the group A′ includes, but is not limited to 3-trifluoromethylbenzylamino, morpholin-4-ylamino, 2-(dimethylamino)ethylamino, 3-(dimethylamino)propylamino, cyclohexylamino, piperidin-1-yl, 2-methoxyethylamino, isopropylamino, isobutylamino, ethylamino, dimethylamino, and methylamino.

In another embodiment, compounds having the following formula: 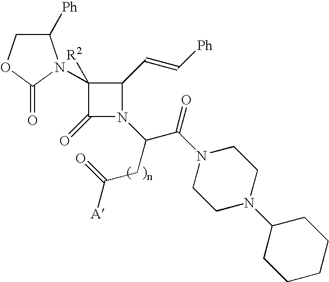
are described, where n and A′ are as described above; R² is illustratively C₁-C₆ alkyl, such as methyl, ethyl, propyl, and the like; haloalkyl, such as trifluoromethyl, chlorodifluoromethyl, tetrafluoroethyl, and the like; C₁-C₄ alkoxy, such as methoxy, ethoxy, and the like; haloalkoxy, such as trifluoromethoxy, chlorodifluoromethoxy, tetrafluoroethoxy, and the like; C₁-C₄ alkylthio, such as methylthio, ethylthio, and the like; cyano; formyl; and halo, such as fluoro and chloro.

It is understood that the above formula represents 16 different stereoisomeric configurations. It is appreciated that certain stereoisomers may be more biologically active than others. Therefore, the above formula contemplates herein all possible stereoisomers, as well as various mixtures of each stereoisomer. Illustratively, the following pair of enantiomers: 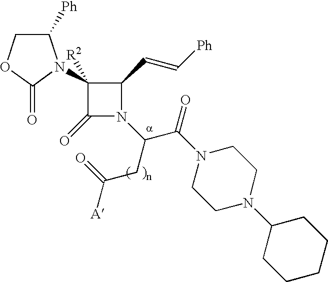
is described, where the sterochemistry at the “α” carbon is either (R) or (S).

In one aspect, the group A′ includes, but is not limited to benzylamino, (2-methylbenzyl)amino, (3-methylbenzyl)amino, (4-methylbenzyl)amino, α-methylbenzyl)amino, N-benzyl-N-methylamino, N-benzyl-N-(t-butyl)amino, N-benzyl-N-butylamino, (3,5-dimethylbenzyl)amino, (2-phenylethyl)amino, dimethylamino, (3-trifluoromethoxybenzyl)amino, (3,4-dichlorobenzyl)amino, (3,5-dichlorobenzyl)amino, (2,5-dichlorobenzyl)amino, (2,3-dichlorobenzyl)amino, (2-fluoro-5-trifluoromethylbenzyl)amino, (4-fluoro-3-trifluoromethylbenzyl)amino, (3-fluoro-5-trifluoromethylbenzyl)amino, (2-fluoro-3-trifluoromethylbenzyl)amino, (4-chloro-3-trifluoromethylbenzyl)amino, indan-1-ylamino, 4-(2-hydroxybenzimidazol-1-yl)-piperidin-1-yl, 3(8)-(tert-butylaminocarbonyl)-1,2,3,4-tetrahydroisoquinolin-2-yl, (3,3-dimethylbutyl)amino, 4-hydroxy-4-phenylpiperidin-1-yl, (cyclohexylmethyl)amino, (2-phenoxyethyl)amino, 3,4-methylenedioxybenzylamino, 4-benzylpiperidin-1-yl, and (3-trifluoromethylphenyl)amino.

In another embodiment, compounds having the formula: 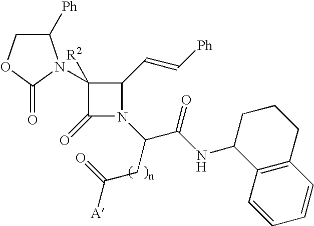
are described, where n and A′ are as described above; R² is illustratively C₁-C₆ alkyl, such as methyl, ethyl, propyl, and the like; haloalkyl, such as trifluoromethyl, chlorodifluoromethyl, tetrafluoroethyl, and the like; C₁-C₄ alkoxy, such as methoxy, ethoxy, and the like; haloalkoxy, such as trifluoromethoxy, chlorodifluoromethoxy, tetrafluoroethoxy, and the like; C₁-C₄ alkylthio, such as methylthio, ethylthio, and the like; cyano; formyl; and halo, such as fluoro and chloro.

It is understood that the above formula represents 32 different stereoisomeric configurations. It is appreciated that certain stereoisomers may be more biologically active than others. Therefore, the above formula contemplates herein all possible stereoisomers, as well as various mixtures of each stereoisomer. Illustratively, the following stereosiomer: 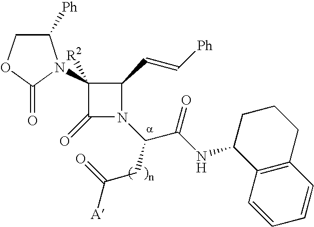
is described.

In another embodiment, compounds having the formula: 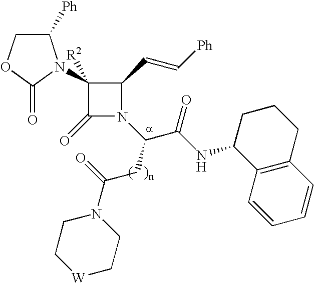
are described, where n is as described above; R² is illustratively C₁-C₆ alkyl, such as methyl, ethyl, propyl, and the like; haloalkyl, such as trifluoromethyl, chlorodifluoromethyl, tetrafluoroethyl, and the like; C₁-C₄ alkoxy, such as methoxy, ethoxy, and the like; haloalkoxy, such as trifluoromethoxy, chlorodifluoromethoxy, tetrafluoroethoxy, and the like; C₁-C₄ alkylthio, such as methylthio, ethylthio, and the like; cyano; formyl; and halo, such as fluoro and chloro; W is either carbon or ntrogen, each optionally substituted with a carbocyclyl substituent, such as cyclopentyl, cyclohexyl, and the like, or an an heterocyclyl substituent, such as pyrollidinyl, piperidinyl, and the like; and the sterochemistry at the “α” carbon is either (R) or (S). The following compounds: 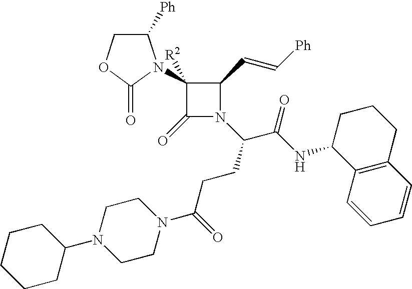
are illustrative of this embodiment, where R² is methyl, methoxy, methylthio, trifluoromethyl, cyano, or fluoro.

In another embodiment, compounds having the following formula: 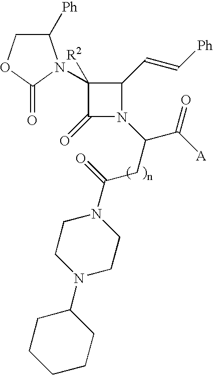
are described, where n and A are as described above; R² is illustratively C₁-C₆ alkyl, such as methyl, ethyl, propyl, and the like; haloalkyl, such as trifluoromethyl, chlorodifluoromethyl, tetrafluoroethyl, and the like; C₁-C₄ alkoxy, such as methoxy, ethoxy, and the like; haloalkoxy, such as trifluoromethoxy, chlorodifluoromethoxy, tetrafluoroethoxy, and the like; C₁-C₄ alkylthio, such as methylthio, ethylthio, and the like; cyano; formyl; and halo, such as fluoro and chloro.

It is understood that the above formula represents 16 different stereoisomeric configurations. It is appreciated that certain stereoisomers may be more biologically active than others. Therefore, the above formula contemplates herein all possible stereoisomers, as well as various mixtures of each stereoisomer. Illustratively, the following pair of enantiomers: 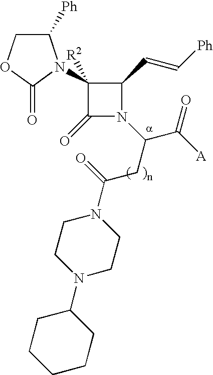
is described, where the sterochemistry at the “α” carbon is either (R) or (S).

In one aspect, the group A′ includes, but is not limited to (3-trifluoromethoxybenzyl)amino, (3,4-dichlorobenzyl)amino, (3,5-dichlorobenzyl)amino, (2,5-dichlorobenzyl)amino, (2,3-dichlorobenzyl)amino, (2-fluoro-5-trifluoromethylbenzyl)amino, (4-fluoro-3-trifluoromethylbenzyl)amino, (3-fluoro-5-trifluoromethylbenzyl)amino, (2-fluoro-3-trifluoromethylbenzyl)amino, (4-chloro-3-trifluoromethylbenzyl)amino, (2-trifluoromethylbenzyl)amino, (3-methoxybenzyl)amino, (3-fluorobenzyl)amino, (3,5-difluorobenzyl)amino, (3-chloro-4-fluorobenzyl)amino, (3-chlorobenzyl)amino, [3,5-bis(trifluoromethyl)benzyl]amino, (3-nitrobenzyl)amino, (3-bromobenzyl)amino, benzylamino, (2-methylbenzyl)amino, (3-methylbenzyl)amino, (4-methylbenzyl)amino, (α-methylbenzyl)amino, (N-methylbenzyl)amino, (N-tert-butylbenzyl)amino, (N-butylbenzyl)amino, (3,5-dimethylbenzyl)amino, (2-phenylethyl)amino, (3,5-dimethoxybenzyl)amino, (1R)-(3-methoxyphenyl)ethylamino, (1S)-(3-methoxyphenyl)ethylamino, (α,α-dimethylbenzyl)amino, N-methyl-N-(3-trifluoromethylbenzyl)amino, [(S)-α-methylbenzyl]amino, (1-phenylcycloprop-1yl)amino, (pyridin-2-ylmethyl)amino, (pyridin-3-ylmethyl)amino, (pyridin-4-ylmethyl)amino, (fur-2-ylmethyl)amino, [(5-methylfur-2-yl)methyl]amino, (thien-2-ylmethyl)amino, [(S)-1,2,3,4-tetrahydro-1-naphth-1-yl]amino, [(R)-1,2,3,4-tetrahydro-1-naphth-1-yl]amino, (indan-1-yl)amino, (1-phenylcyclopent-1-yl)amino, (α,α-dimethyl-3,5-dimethoxybenzyl)amino, (2,5-dimethoxybenzyl)amino, (2-methoxybenzyl)amino, and (α,α,2-trimethylbenzyl)amino.

The above compounds may be prepared by reacting a suitably substituted compound having the formula: 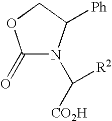
with a compound having the formula: 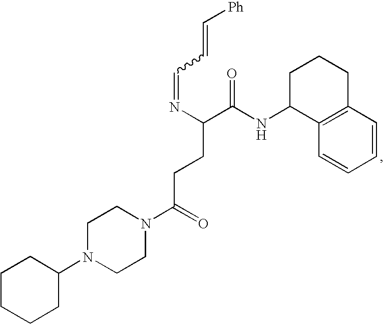
where R² is as defined above for formula I. It is appreciated that any desired stereochemical configuration of these compounds may be prepared using this process by selecting the desired configuration at each chiral center noted above. Such a selection may be accomplished by using optically pure starting materials, or by separating mixtures of optical isomers at convenient times during the syntheses of the two foregoing formulae using standard techniques.

Further illustrative classes of compounds are described by compounds having formula III: 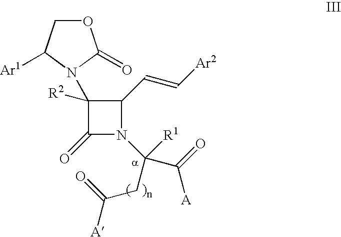
where R¹, R², R⁴, A, and A′ are as defined above, and Ar¹ and Ar² are each an optionally substituted aryl group, each independently selected.

In one aspect, Ar¹ is optionally substituted phenyl, optionally substituted pyridinyl, optionally substituted furyl, or optionally substituted thienyl;

R² is hydrogen;

A is XNH—;

A′ is X′NH—;

A′ is R^{5′}X′N—;

n is 0, 1, or 2;

X is optionally substituted aryl(C₁-C₄ alkyl), and amyl is substituted phenyl;

A′ is R^{6′}O—;

R^{6′} is C₁-C₆ alkyl;

X′ is R^{7′}R^{8′}N—;

X′ is optionally substituted aryl(C₁-C₄ alkyl);

X′ is an heterocycle Y′;

R^{5′} and X′ are taken together with the attached nitrogen atom to form piperidinyl, piperazinyl, or homopiperazinyl; where said piperidinyl, piperazinyl, or homopiperazinyl is optionally substituted with C₁-C₆ alkyl, C₃-C₈ cycloalkyl, an heterocycle Y′, optionally substituted aryl(C₁-C₄ alkyl), R⁷R⁸N—, R⁷R⁸N—(C₁-C₄ alkyl), or R⁷R⁸N—C(O)—(C₁-C₄ alkyl);

R^{8′} is C₁-C₆ alkyl, C₃-C₈ cycloalkyl, optionally substituted aryl, optionally substituted aryl(C₁-C₄ alkyl); and

R^{7′} and R^{8′} are taken together with the attached nitrogen atom to form an heterocycle selected from the group consisting of pyrrolidinyl, piperidinyl, morpholinyl, and piperazinyl; where said piperazinyl is optionally substitued at the 4-position with C₁-C₄ alkyl.

In another embodiment, the compounds of formula I include a basic amino group. Such amines are capable of forming salts with a variety of inorganic and organic acids to form pharmaceutically acceptable acid addition salts. It is appreciated that in cases where compounds of formula I are oils rather than solids, those compounds capable of forming addition salts that are solid will ease the handling and administration of the compounds described herein. Acids commonly employed to form such salts are inorganic acids such as hydrochloric acid, hydrobromic acid, hydroiodic acid, sulfuric acid, phosphoric acid, and the like, and organic acids, such as p-toluenesulfonic acid, methanesulfonic acid, oxalic acid, p-bromophenylsulfonic acid, carbonic acid, succinic acid, citric acid, benzoic acid, acetic acid, and the like. Examples of such pharmaceutically acceptable salts thus are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, β-hydroxybutyrate, glycollate, tartrate, methanesulfonate, propanesulfonate, naphthalene-1-sulfonate, naphthalene-2-sulfonate, mandelate and the like. Preferred pharmaceutically acceptable salts are those formed with hydrochloric acid, trifluoroacetic acid, maleic acid or fumaric acid.

The compounds described herein are useful in methods for antagonism of the vasopressin V₁ₐ receptor. Such antagonism is useful in treating a variety of disorders and diseases that have been linked to this receptor in mammals. Illustratively, the mammal to be treated by the administration of compounds described herein is human.

The 2-(azetidinon-1-yl)alkanedioic acid esters and amides of formulae I II, and III may be prepared by syntheses known in the art, as well as by the new methods described herein. As illustrated for compounds of formula I, the 2-(azetidinon-1-yl)alkanedioic acid esters described herein are obtainable by the 2+2 cycloaddition of an appropriately substituted acetic acid derivative (i), and an imine ester (ii) upon treatment with a base in an appropriately selected solvent, as described in Synthetic Scheme I, where Z is a leaving group, and the integer n, and the moieties A, A′, R¹, R², R³, and R⁴ are as previously described. The term “leaving group” as used hereinafter refers to a subsitutent, such as halo, acyloxy, benzoyloxy and the like, present on an activated carbon atom that may be replaced by a nucleophile. The chemistry described in Synthetic Scheme I is applicable to imines (ii) bearing ester, thioester, or amide moieties.

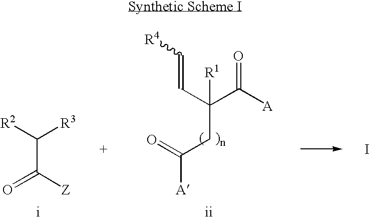

The preparation of the appropriate imines (ii) and most of the required acetyl halides or anhydrides (i), as well as the cycloaddition procedure, are generally described in U.S. Pat. Nos. 4,665,171 and 4,751,299, the desclosure of which are hereby incorporated by reference. The analogous synthesis of compounds of formulae I and III may be accomplished by this process using the appropriate alkoxy-substituted amino acid imines.

Those compounds of formulae I, II, and III of the invention requiring R³ to be a 4-substituted oxazolidin-2-on-3-yl or 1,4,5-trisubstituted imidazolidin-2-on-3-yl are prepared from the corresponding (4-substituted oxazolidin-2-on-3-yl) or (1,4,5-trisubstituted imidazolidin-2-on-3-yl)acetyl halide or anhydride. The acid halide or anhydride is available from an appropriately substituted glycine. The glycine is first converted to the carbamate and then reduced to provide the corresponding alcohol. The alcohol is then cyclized to the 4-substituted oxazolidin-2-one, which is subsequently N-alkylated with a haloacetic acid ester. The ester is hydrolyzed, and the resulting acid is converted to the acetyl halide or anhydride (i).

Those compounds of the invention requiring R³ to be 2,5-disubstituted oxazolidin-4-on-3-yl or 1,2,5-trisubstituted imidazolidin-4-on-3-yl are prepared from the corresponding (2,5-disubstituted oxazolidin-4-on-3-yl) or (1,2,5-trisubstituted imidazolidin-4-on-3-yl)acetyl chlorides or anhydrides respectively. The chemistry to prepare these reagents is described in U.S. Pat. No. 4,772,694, hereby incorporated by reference. Briefly, the required oxazolidinone or imidazolidinone is obtained from an α-hydroxyacid or an α-aminoacid, respectively. The imidazolones are prepared by converting the α-aminoacid, (R¹¹)—CH(NH₂)CO₂H, to an amino-protected amide and then condensing the amide with an aldehyde, (R¹⁰)—CHO, in the presence of an acid to form the 3-protected imidazolidin-4-one, where R¹⁰ and R¹¹ are as defined above. The 1-position may be functionalized with an appropriate reagent to introduce R¹² and the 3-position deprotected, where R¹² is as defined above. The imidazolidin-4-one ring is then alkylated with a haloacetic acid ester, the ester deesterified, and the resulting acetic acid converted to the desired acid halide or anhydride (i). The required oxazolidinones are prepared in an analogous manner from the corresponding α-hydroxyacid, (R¹¹)—CH(OH)CO₂H.

Those compounds of the invention requiring R³ to be succinimido are prepared from the corresponding 2-(succinimido)acetyl halide or anhydride. The chemistry to prepare these reagents is described in U.S. Pat. No. 4,734,498, hereby incorporated by reference. Briefly, these reagents are obtained from tartaric acid or, when one of R¹⁰ and R¹¹ is hydrogen, from malic acid. Tartaric acid is acylated or O-alkylated, the corresponding diacyl or di-O-alkyl tartaric acid is treated with an acid anhydride to form the succinic anhydride, and reaction of this succinic anhydride with an ester of glycine to form first the noncyclic half amide ester which is then cyclized to the 3,4-disubstituted succinimidoacetic acid ester. The ester group is deesterified and the resulting acid converted to the corresponding acid halide or anhydride (i). The mono-substituted succinimidoacetyl halide or anhydride is obtained with malic acid via succinic anhydride formation followed by succinimide formation as described above.

Those compounds of the invention requiring R³ to be an N-substituted amine or an N′-substituted urea may be prepared from the corresponding phthalimido protected 3-amino analogs. The phthalimide protecting group may be removed using conventional procedures, such as by treatment with hydrazine, and the like. Once liberated, the amine may be alkylated with any one of a variety of alkyl and cycloalkyl halides and sulfates, such as methyl iodide, isopropylbromide, diethyl sulfate, cyclopropylmethylbromide, cyclopentyliodide, and the like. Such amines may also be acylated with acid halides, acid anhydrides, isocyanates, isothiocyanates, such as acetyl chloride, propionic anhydride, methylisocyanate, 3-trifluoromethylphenylisothiocyanate, and the like.

The bases to be used in Synthetic Scheme I include, among others, aliphatic tertiary amines, such as trimethylamine and triethylamine, cyclic tertiary amines, such as N-methylpiperidine and N-methylmorpholine, aromatic amines, such as pyridine and lutidine, and other organic bases such as 1,8-diazabicyclo[5,4,0]undec-7-ene (DBU).

The solvents useful for reactions described in Synthetic Scheme I include, among others, dioxane, tetrahydrofuran, diethyl ether, ethyl acetate, dichloromethane, chloroform, carbon tetrachloride, benzene, toluene, acetonitrile, dimethyl sulfoxide and N,N-dimethylformamide.

Alternatively, the compounds of formulae I, II, and III may be prepared via N—C(4) cyclization, as illustrated for compounds of formula I in Synthetic Scheme II, via cyclizatoin of β-hydroxy amides iii, where R¹, R², R³, R⁴, A, and A′ are as defined previously, according to the procedure of Townsend and Nguyen in J. Am. Chem. Soc. 1981, 103, 4582, and Miller and Mattingly in Tetra. 1983, 39, 2563, the disclosures of which are incorporated herein by reference. The analogous synthesis of compounds of formulae II and III may be accomplished by cyclizatoin of β-hydroxy amides of alkoxy-substituted amino acids.

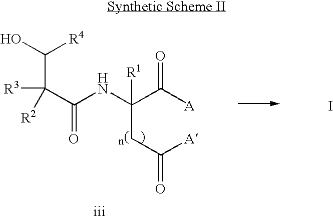

The azetidinone ring may also be prepared with a deficit of substituents R², R³, R⁴, or the R¹-substituted N-alkanedioic acid or alkoxyalkanoic acid moiety, but possessing substituents capable of being elaborated through subsequent chemical transformation to such groups described for compounds of formulae I and II. In general, azetidinones may be prepared via N—C(4) cyclization, such as the cyclization of acylhydroxamates iv to azetidinone intermediates v, as depicted in Scheme III, where R¹, R², R³, R⁴, A, and A′ are as defined above, according to the procedure of Mattingly et al. in J. Am. Chem. Soc. 1979, 101, 3983 and Accts. Chem. Res. 1986, 19, 49, the disclosures of which are incorporated herein by reference. It is appreciated that other hydroxamates, such as alkylhydroxamates, aryl hydroxamates, and the like, are suitable for carrying out the cyclization.

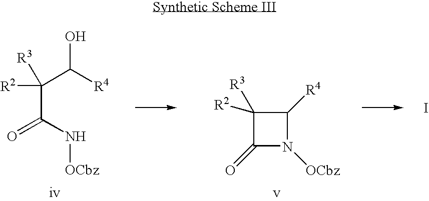

Subsequent chemical transformation of the acyloxyazetidinone v to introduce for example an R¹-substituted alkanedioic acid moiety using conventional procedures will illustratively provide compounds of formula I. The analogous synthesis of compounds of formulae II and III may be accomplished by this process using an appropriate R¹-substituted alkoxyalkanoic acid.

An alternative cyclization to form intermediate azetidinones, which may be further elaborated to compounds of formulae I, II, and III may occur by oxidative cyclization of acylhydroxamates vi to intermediate azetidinones vii, as illustrated in Synthetic Scheme IV, where R² and R³ are as defined above and L is a leaving group such as halide, according to the procedure of Rajendra and Miller in J. Org. Chem. 1987, 52, 4471 and Tetrahedron Lett. 1985, 26, 5385, the disclosures of which are incorporated herein by reference. The group R in Scheme IV represents an alkyl or aryl moiety selected to provide R⁴, as defined above, upon subsequent transformation. For example, R may be the group ArCH₂— where Ar is an optionally substituted aryl group, as in vii-a, such that oxidative elimination of HBr will provide the desired R⁴, such as a styryl group, as in vii-b. It is appreciated that elaboration of R to R⁴ is not necessarily performed immediately subsequent to the cyclization and may be performed conveniently after other steps in the synthesis of compounds of formulae I, II, and III. It is further appreciated that alternatives to the acylhydroxamates shown, such as alkylhydroxamates, aryl hydroxamates, and the like, are suitable for carrying out the cyclization.

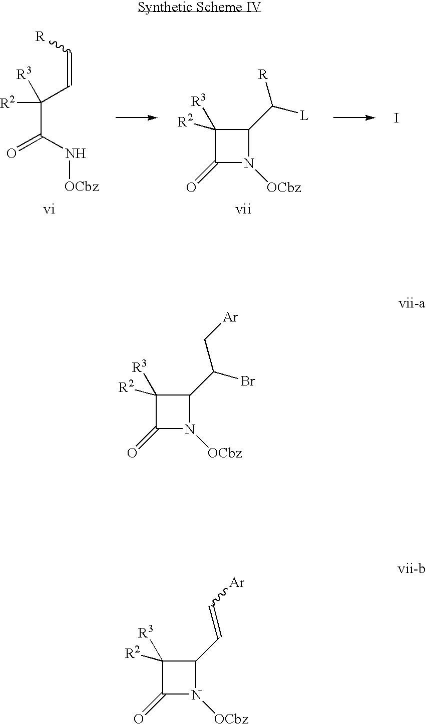

Other useful intermediates, such as the azetidinone-4-carboxaldehyde viii illustrated in Synthetic Scheme V for preparing compounds of formulae I, II or III may be further elaborated to 4-(R⁴)-substituted azetidinones via an olefination reaction. The groups R¹, R², and R³ are as defined above, and the group R in Scheme V is selected such that upon successful olefination of the carboxaldehyde the resulting group R—CHCH— corresponds to the desired alkyl or aryl moiety R⁴, as defined above. Such olefination reactions may be accomplished by any of the variety of known procedures, such as by Wittig olefination, Peterson olefination, and the like. Synthetic Scheme V illustrates the corresponding Wittig olefination with phosphorane ix. The analogous synthesis of compounds of formulae II and III may be accomplished by this process using an appropriate alkoxy-substituted azetidinone-4-carboxaldehyde derivative.

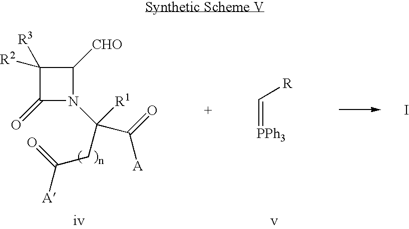

Still other useful intermediates, such as the azetidinonyl acetic acid derivatives x, may be converted into compounds of formulae I, II, and III, as illustrated for the synthesis of compounds of formula I in Synthetic Scheme VI, where R¹, R², R³, R⁴, A, A′ and n are as defined above. Introduction of the R¹ moiety, and a carboxylic acid derivative A′—C(O)—(CH₂)ₙ— for compounds of formula I, may be accomplished by alkylation of the anion of x.

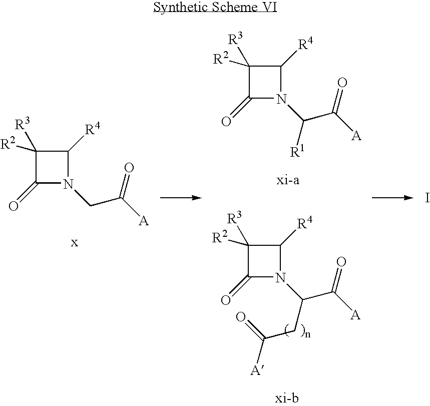

Acetic acid derivative x is deprotonated and subsequently alkylated with an alkyl halide corresponding to R¹-Z, where Z is a leaving group, to provide intermediate xi-a. Illustratively, the anion of xi-a may be alkylated with a compound Z′-(CH₂)ₙCOA′, where Z′ is a leaving group, to provide compounds of formula I.

Alternatively, acetic acid derivative x is deprotonated and subsequently alkylated with a compound Z′-(CH₂)ₙCOA′, where Z′ is a leaving group, to provide intermediate xi-b. Illustratively, the anion of xi-b may be alkylated with an alkyl halide corresponding to R¹-Z, where Z is a leaving group, to provide compounds of formula I. It is appreciated that the order of introduction of either the substituent R¹ or the acid derivative —(CH₂)ₙCOA′, may be dictated by steric or electronic considerations, synthetic convenience, or the availability of certain starting materials, and such order of introduction may be different for each compound of formulae I, II, or III.

A solution of the 2-(3,4-disubstituted azetidin-2-on-1-yl)acetic acid derivative x or xi in an appropriate solvent, such as tetrahydrofuran, dioxane, or diethyl ether, is treated with a non-nucleophilic base to generate the anion of x or xi, respectively. Suitable bases for this transformation include lithium diisopropylamide, lithium 2,2,6,6-tetramethylpiperidinamide, or lithium bis(trimethylsilyl)amide. The anion is then reacted with an appropriate electrophile to provide the desired compounds. Illustrative electrophiles represented by the formulae R¹-Z, R^{5′}X′N—C(O)—(CH₂)ₙ-Z, or R^{6′}O—C(O)—(CH₂)ₙ-Z provide the corresponding compounds xi or I, respectively. The analogous synthesis of compounds of formulae II and III may be accomplished by this process by using the appropriate electrophile.

As discussed above, the compounds prepared as described in Synthetic Schemes I, II, III, V, and VI may be pure diastereomers, mixtures of diastereomers, or racemates. The actual stereochemical composition of the compound will be dictated by the specific reaction conditions, combination of substituents, and stereochemistry or optical activity of the reactants employed. It is appreciated that diasteromeric mixtures may be separated by chromatography or fractional crystallization to provide single diastereomers if desired, using standard methods. Particularly, the reactions described in Synthetic Schemes III, IV, and VI create a new chiral center at the carbon bearing R¹, except when n=0 and A=A′.

Compounds of formula I which are 2-(3,4-disubstituted azetidin-2-on-1-yl)alkanedioic acid half-esters, such as compounds I-a where A′ is R^{6′}O—, while useful vasopressin V₁ₐ agents in their own right, may also be converted to the corresponding half-carboxylic acids xii, where the integer n and the groups R¹, R², R³, R⁴, R^{5′}, R^{6′}, A, and X′ are as previously defined, as illustrated in Synthetic Scheme VII. These intermediates are useful for the preparation of other compounds of the invention, such as I-b where A′ is R^{5′}X′N—. It is appreciated that the transformation illustrated in Synthetic Scheme VII is equally applicable for the preparation of compounds I where A′ is X′NH— or where a different R^{6′}O— is desired.

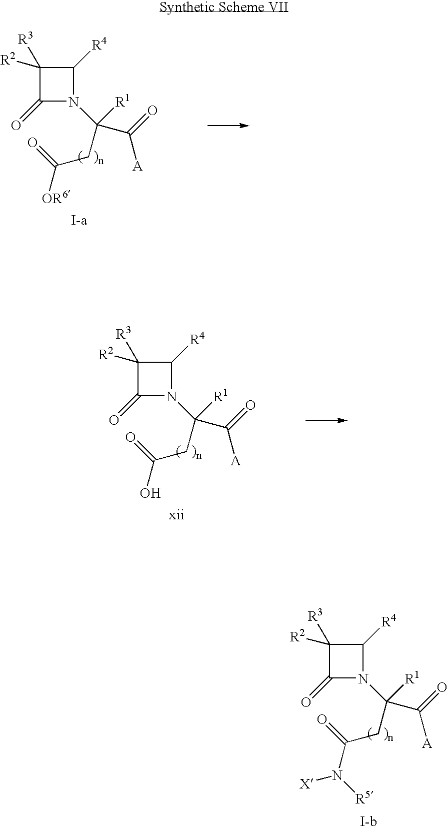

The requisite carboxylic acid xii may be prepared from the corresponding ester via saponification under standard conditions by treatment with hydroxide followed by protonation of the resultant carboxylate anion. Where R^{6′} is tert-butyl, the ester I-a may be dealkylated by treatment with trifluoroacetic acid. Where R^{6′} is benzyl, the ester I-a may be dealkylated either by subjection to mild hydrogenolysis conditions, or by reaction with elemental sodium or lithium in liquid ammonia. Finally, where R^{6′} is 2-(trimethylsilyl)ethyl, the ester I-a may be deprotected and converted into the corresponding acid xii by treatment with a source of fluoride ion, such as tetrabutylammonium fluoride. The choice of conditions is dependent upon the nature of the R^{6′} moiety and the compatability of other functionality in the molecule with the reaction conditions.

The carboxylic acid xii is converted to the corresponding amide I-b under standard conditions. The acid may be first converted to the corresponding acid halide, preferably the chloride or fluoride, followed by treatment with an appropriate primary or secondary amine to provide the corresponding amide. Alternatively, the acid may be converted under standard conditions to a mixed anhydride. This is typically accomplished by first treating the carboxylic acid with an amine, such as triethylamine, to provide the corresponding carboxylate anion. This carboxylate is then reacted with a suitable haloformate, for example benzyl chloroformate, ethyl chloroformate or isobutylchloroformate, to provide the corresponding mixed anhydride. This anhydride may then be treated with an appropriate primary or secondary amine to provide the desired amide. Finally, the carboxylic acid may be treated with a typical peptide coupling reagent such as N,N′-carbonyldiimidazole (CDI), N,N′-dicyclohexylcarbodiimide (DCC) and 1-(3-dimethylaminopropyl)-3-ethylcarbodiimide hydrochloride (EDC), followed by the appropriate amine of formula R⁵XNH. A polymer-supported form of EDC has been described in Tetrahedron Letters, 34(48):7685 (1993), the disclosure of which is incorporated herein by reference, and is useful for the preparation of the compounds of the present invention. It is appreciated that substituting an appropriate amine with an appropriate alcohol in the synthethic scheme presented above will provide the esters of the invention, e.g. analogs of I-a with a different ester R^{6′}O—.

The carboxylic acid may alternatively be converted into the corresponding tert-butyl ester via treatment of the acid with an acid catalyst, such as concentrated sulfuric acid, and the like, and with isobutylene in a suitable solvent, such as dioxane, and the like. The reaction is preferably carried out under pressure in an appropriate vessel, such as a pressure bottle, and the like. Reaction times of about 18 hours are not uncommon. The desired ester may be be isolated from the organic layer after partitioning the reaction mixture between a suitable organic solvent, such as ethyl acetate, and the like, and a basic aqueous layer, such as cold 1N sodium hydroxide, and the like.

It is appreciated that the transformation illustrated in Synthetic Scheme VII may also be used to convert in an analogous fashion, the half-ester I where A is R⁶O— to the corresponding acid and subsequently into derivatives I where A is XNH—, R⁵XN—, or a different R⁶O—. Finally, it is appreciated that the general synthetic strategy represented by the transformation in Synthetic Scheme VII is equally applicable to changing the carboxylic acid derivatives in compounds of formulae II and III.

Compounds of formulae I, II, and III where R⁴ includes an ethenyl or ethynyl spacer, such as for example, compounds I-c and I-d, respectively, may be converted into the corresponding arylethyl derivatives, compounds I-e, via reduction, as illustrated for compounds of formula I in Synthetic Scheme VIII. Conversion is accomplished by catalytic hydrogenation, and other like reductions, where the integer n and the groups R¹, R², R³, A, and A′ are as previously defined. The corresponding compounds of formulae II and III may also be converted from ethyne and ethene precursors in an analogous fashion. The moiety R depicted in Scheme VIII is chosen such that the substituent R—CC—, R—CHCH—, or R—CH₂CH₂— corresponds to the desired R⁴ of formulae I, II, or III as defined above.

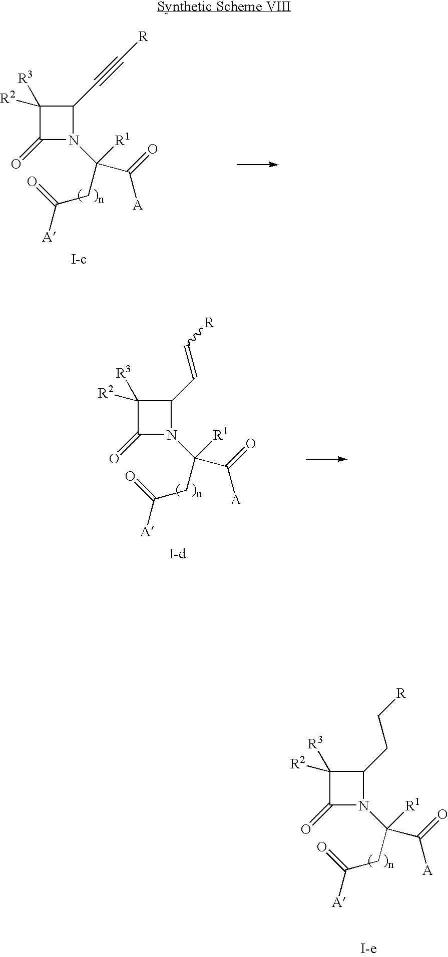

The hydrogenation of the triple or double bond proceeds readily over a precious metal catalyst, such as palladium on carbon. The hydrogenation solvent may consist of a lower alkanol, such as methanol or ethanol, tetrahydrofuran, or a mixed solvent system of tetrahydrofuran and ethyl acetate. The hydrogenation may be performed at an initial hydrogen pressure of about 20-80 p.s.i., preferably about 50-60 p.s.i., at a temperature of about 0-60° C., preferably within the range of from ambient temperature to about 40° C., for about 1 hour to about 3 days.

Alternatively, the ethynyl spacer of compound I-c may be selectively reduced to the ethenyl spacer of compound I-d using poisoned catalyts, such as Pd on BaSO₄, Lindlar's catalyst, and the like. It is appreciated that either the Z or E double bond geometry of compound I-d may be advantageously obtained by the appropriate choice of reaction conditions. Alternatively, a mixture of double bond geometries may be prepared. The analogous synthesis of compounds of formulae II and III may be accomplished by this process.

Compounds of formulae I, II, and III where R³ is phthalimido are conveniently treated with hydrazine or a hydrazine derivative, for example methylhydrazine, to prepare the corresponding 2-(3-amino-4-substituted azetidin-2-on-1-yl)alkanedioic acid derivatives xiii, as illustrated in Synthetic Scheme IX for compounds of formula I, where the integer n, and the groups R¹, R², R⁴, R¹², A, and A′ are as previously defined. Intermediate xiii may then be treated with an appropriate alkylating or acylating agent to prepare the corresponding amines or amides I-g, or alternatively intermediates xiii may be treated with an appropriate isocyanate to prepare the corresponding ureas I-h.

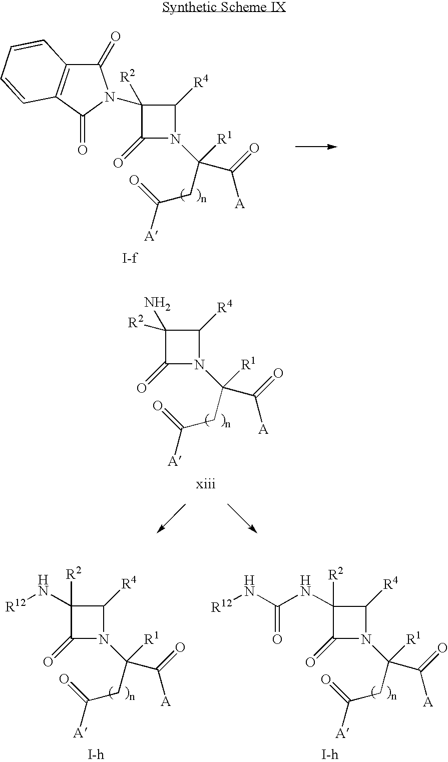

The ureas I-h are prepared by treating a solution of the appropriate amine xiii in a suitable solvent, such as chloroform or dichloromethane, with an appropriate isocyanate, R¹²NCO. If necessary, an excess of the isocyanate is employed to ensure complete reaction of the starting amine. The reactions are performed at about ambient temperature to about 45° C., for from about three hours to about three days. Typically, the product may be isolated by washing the reaction with water and concentrating the remaining organic components under reduced pressure. When an excess of isocyanate has been used, however, a polymer bound primary or secondary amine, such as an aminomethylated polystyrene, may be conveniently added to facilitate removal of the excess reagent. Isolation of products from reactions where a polymer bound reagent has been used is greatly simplified, requiring only filtration of the reaction mixture and then concentration of the filtrate under reduced pressure.

The substituted amines and amides I-g are prepared by treating a solution of the appropriate amine xiii in a suitable solvent, such as chloroform or dichloromethane, with an appropriate acylating or alkylating agent, R¹²—C(O)Z or R¹²-Z, respectively. If necessary, an excess of the acylating or alkylating agent is employed to ensure complete reaction of the starting amine. The reactions are performed at about ambient temperature to about 45° C., for from about three hours to about three days. Typically, the product may be isolated by washing the reaction with water and concentrating the remaining organic components under reduced pressure. When an excess of the acylating or alkylating agent has been used, however, a polymer bound primary or secondary amine, such as an aminomethylated polystyrene, may be conveniently added to facilitate removal of the excess reagent. Isolation of products from reactions where a polymer bound reagent has been used is greatly simplified, requiring only filtration of the reaction mixture and then concentration of the filtrate under reduced pressure. The analogous synthesis of compounds of formulae II and III may be accomplished by this process.

The following preparations and examples further illustrate the synthesis of the compounds described herein and are not intended to limit the scope of the invention in any way. Unless otherwise indicated, all reactions were performed at ambient temperature, and all evaporations were performed in vacuo. All of the compounds described below were characterized by standard analytical techniques, including nuclear magnetic resonance spectroscopy (¹H NMR) and mass spectral analysis (MS).

### EXAMPLES

### Example 1

### Methyl (4(S)-phenyloxazolidin-2-on-3-yl)acetate

A solution of (4(S)-phenyloxazolidin-2-on-3-yl)acetic acid (1 g, 4.52 mmol) (prepared according to Evans in U.S. Pat. No. 4,665,171) in 20 mL of anhydrous methanol was treated hourly with 5 equivalents of acetyl chloride, for a total of 20 equivalents. The resulting solution was stirred overnight. The residue obtained after evaporation of the MeOH was redissolved in 30 mL of CH₂Cl₂ and treated with 50 mL of saturated aqueous Na₂CO₃. The organic layer was evaporated and dried (MgSO₄) to yield the title compound as a colorless oil (1.001 g, 94%); ¹H NMR (CDCl₃) δ 3.37 (d, J=18.0 Hz, 1H), 3.69 (s, 3H), 4.13 (t, J=8.3 Hz, 1H), 4.28 (d, J=18.0 Hz, 1H), 4.69 (t, J=8.8 Hz, 1H), 5.04 (t, J=8.4 Hz, 1H), 7.26-7.29 (m, 2H), 7.36-7.42 (m, 3H).

### Example 2

### Methyl 2-(4(S)-phenyloxazolidin-2-on-3-yl)propanoate

A solution of methyl (4(S)-phenyloxazolidin-2-on-3-yl)acetate (1 g, 4.25 mmol) in 10 mL of anhydrous THF at −78° C. was treated with 4.68 mL (4.68 mmol) of a 1 M solution of lithium bis(trimethylsilyl)amide in THF. The reaction mixture was stirred for 1 h. at about −70° C. before adding MeI (1.59 mL, 25.51 mmol). Upon complete conversion of the azetidinone, the reaction was quenched with saturated aqueous NH₄Cl and partitioned between EtOAc and water. The organic layer was washed sequentially with saturated aqueous sodium bisulfite, and saturated aqueous NaCl. The resulting organic layer was dried (MgSO₄) and evaporated to afford the title compound (a mixture of diasteromers) as a white solid (1.06g, 93%); ¹H NMR (CDCl₃) δ 1.07/1.53 (d/d, J=7.5 Hz, 3H), 3.59/3.74 (s/s, 3H), 3.85/4.48 (q/q, J=7.5 Hz, 1H), 4.10-4.14 (m, 1H), 4.60-4.64/4.65-4.69 (m/m, 1H), 4.88-4.92/4.98-5.02 (m/m, 1H), 7.24-7.40 (m, 5H).

### Example 3

### 2-(4(S)-Phenyloxazolidin-2-on-3-yl)propanoic acid

To a solution of methyl 2-(4(S)-phenyloxazolidin-2-on-3-yl)propanoate (1 g, 4.01 mmol) in 35 mL of MeOH was added, at 0° C., 14.3 mL (12.04 mmol) of a 0.84 M solution of LiOH in water. The reaction mixture was then stirred for 3 h. at ambient temperature. Upon complete hydrolysis of the azetidinone, the MeOH was removed by evaporation, the crude residue dissolved in CH₂Cl₂ and treated with saturated aqueous NaCl. The resulting organic layer was dried (MgSO₄) and evaporated to afford the title compound (racemic mixture) as a white solid (0.906 g, 96%); ¹H NMR (CDCl₃) δ 1.13/1.57 (d/d, J=7.5 Hz, 3H), 3.75/4.50 (q/q, J=7.5 Hz, 1H), 4.10-4.16 (m, 1H), 4.62-4.72 (m, 1H), 4.92-5.03 (m, 1H), 7.32-7.43 (m, 5H).

### Example 4

### 2-(4(S)-Phenyloxazolidin-2-on-3-yl propanoyl chloride

A solution of 1 equivalent of Example 3 and 1.3 equivalent of oxalyl chloride in 200 mL CH₂Cl₂ (150 mL/g of propanoic acid derivative) was treated with a catalytic amount of anhydrous DMF (85 μL/mmole of propanoic acid derivative) resulting in vigorous gas evolution. After 45 min., all gas evolution had ceased and the reaction mixture was concentrated under reduced pressure to provide the title compound as an off-white solid after drying for 2 h under vacuum.

### Example 5

### General Procedure for Amide Formation from an Activated Ester Derivative

N-Benzyloxycarbonyl-L-aspartic acid β-t-butyl ester α-(3-trifluoromethyl)benzylamide.

A solution of N-benzyloxycarbonyl-L-aspartic acid β-t-butyl ester α-N-hydroxysuccinimide ester (1.95 g, 4.64 mmol, Advanced ChemTech) in 20 mL of dry tetrahydrofuran was treated with 0.68 mL (4.74 mmol) of 3-(trifluoromethyl)benzyl amine. Upon completion (TLC, 60:40 hexanes/ethyl acetate), the mixture was evaporated, and the resulting oil was partitioned between dichloromethane and a saturated aqueous solution of sodium bicarbonate. The organic laer was evaporated to give 2.23 g (quantitative yield) of the title compound as a white solid; ¹H NMR (CDCl₃) δ 1.39 (s, 9H), 2.61 (dd, J=6.5 Hz, J=17.2 Hz, 1H), 2.98 (dd, J=3.7 Hz, J=17.0 Hz, 1H), 4.41 (dd, J=5.9 Hz, J=15.3 Hz, 1H), 4.50-4.57 (m, 2H), 5.15 (s, 2H), 5.96-5.99 (m, 1H), 6.95 (s, 1H), 7.29-7.34 (m, 5H), 7.39-7.43 (m, 2H), 7.48-7.52 (m, 2H).

Examples 6-8 were prepared according to the procedure of Example 5, except that N-benzyloxycarbonyl-L-aspartic acid β-t-butyl ester α-N-hydroxysuccinimide ester was replaced by the appropriate amino acid derivative, and 3-(trifluoromethyl)benzyl amine was replaced with the appropriate amine.

### Example 6

### N-Benzyloxycarbonyl-L-aspartic acid β-t-butyl ester α-[4-(2-phenylethyl)]piperazinamide

N-benzyloxycarbonyl-L-aspartic acid β-t-butyl ester α-N-hydroxysuccinimide ester (5.0 g, 12 mmol, Advanced ChemTech) and 4-(phenylethyl)piperazine 2.27 mL (11.9 mmol) gave 5.89 g (quantitative yield) of the title compound as an off-white oil; ¹H NMR (CDCl₃) δ 1.40 (s, 9H), 2.45-2.80 (m, 10H), 3.50-3.80 (m, 4H), 4.87-4.91 (m, 1H), 5.08 (s, 2H), 5.62-5.66 (m, 1H), 7.17-7.33 (m, 10H).

### Example 7

### N-Benzyloxycarbonyl-L-glutamic acid γ-t-butyl ester α-(3-trifluoromethyl)benzylamide

N-benzyloxycarbonyl-L-glutamic acid β-t-butyl ester α-N-hydroxysuccinimide ester (4.83 g, 11.1 mmol, Advanced ChemTech) and 3-(trifluoromethyl)benzylamine) 1.63 mL (11.4 mmol) gave 5.41 g (98%) of the title compound as an off-white solid; ¹H NMR (CDCl₃) δ 1.40 (s, 9H), 1.88-1.99 (m, 1H), 2.03-2.13 (m, 1H), 2.23-2.33 (m, 1H), 2.38-2.47 (m, 1H), 4.19-4.25 (s, 1H), 4.46-4.48 (m, 2H), 5.05-5.08 (m, 2H), 5.67-5.72 (m, 1H), 7.27-7.34 (m, 5H), 7.39-7.43 (m, 2H), 7.48-7.52 (m, 2H).

### Example 8

### N-Benzyloxycarbonyl-L-glutamic acid β-t-butyl ester α-[4-(2-phenylethyl)]piperazinamide

N-benzyloxycarbonyl-L-glutamic acid γ-t-butyl ester α-N-hydroxysuccinimide ester (5.0 g, 12 mmol, Advanced ChemTech) and 4-(phenylethyl)piperazine 2.19 mL (1.5 mmol) gave 5.87 g (quantitative yield) of the title compound as an off-white oil; ¹H NMR (CDCl₃) δ 1.43 (s, 9H); 1.64-1.73 (m, 1H); 1.93-2.01 (m, 1H); 2.23-2.40 (m, 2H); 2.42-2.68 (m, 6H); 2.75-2.85 (m, 2H); 3.61-3.74 (m, 4H); 4.66-4.73 (m, 1H); 5.03-5.12 (m, 2H); 5.69-5.72 (m, 1H); 7.16-7.34 (m, 10H).

### Example 9

### N-[(9H-Fluoren-9-yl methoxycarbonyl]-O-(benzyl)-D-serine t-Butyl ester

N-[(9H-Fluoren-9-yl)methoxycarbonyl]-O-(benzyl)-D-serine (0.710 g, 1.70 mmole) in dichloromethane (8 mL) was treated with t-butyl acetate (3 mL) and concentrated sulfuric acid (40 μL) in a sealed flask at 0° C. Upon completion (TLC), the reaction was quenched with of dichloromethane (10 mL) and saturated aqueous potassium bicarbonate (15 mL). The organic layer was washed with distilled water, and evaporated. The resulting residue was purified by flash column chromatography (98:2 dichloromethane/methanol) to yield the title compound as a colorless oil (0.292 g, 77%); ¹H NMR (CDCl₃) δ 1.44 (s, 9H); 3.68 (dd, J=2.9 Hz, J=9.3 Hz, 1H); 3.87 (dd, J=2.9 Hz, J=9.3 Hz, 1H); 4.22 (t, J=7.1 Hz, 1H); 4.30-4.60 (m, 5H); 5.64-5.67 (m, 1H); 7.25-7.39 (m, 9H); 7.58-7.61 (m, 2H); 7.73-7.76 (m, 2H).

### Example 10

### O-(Benzyl)-D-serine t-Butyl ester

Example 9 (0.620 g, 1.31 mmol) in dichloromethane (5 mL) was treated with tris(2-aminoethyl)amine (2.75 mL) for 5 h. The resulting mixture was washed twice with a phosphate buffer (pH=5.5), once with saturated aqueous potassium bicarbonate, and evaporated to give 0.329 g (quantitative yield) of the title compound as an off-white solid; ¹H NMR (CD₃OD) δ 1.44 (s, 9H); 3.48 (dd, J=J′=4.2 Hz, 1H); 3.61 (dd, J=4.0 Hz, J=9.2 Hz, 1H); 3.72 (dd, J=4.6 Hz, J=9.2 Hz, 1H); 4.47 (d, J=12.0 Hz, 1H); 4.55 (d, J=12.0 Hz, 1H); 7.26-7.33 (m, 5H).

### Example 11

### General Procedure for Amide Formation from a Carboxylic Acid

N-Benzyloxycarbonyl-D-aspartic acid β-t-butyl ester α-(3-trifluoromethyl)benzylamide.

A solution of 1 g (2.93 mmol) of N-benzyloxycarbonyl-D-aspartic acid β-t-butyl ester monohydrate (Novabiochem) in 3-4 mL of dichloromethane was treated by sequential addition of 0.46 mL (3.21 mmol) of 3-(trifluoromethyl)benzylamine, 0.44 g (3.23 mmol) of 1-hydroxy-7-benzotriazole, and 0.62 g (3.23 mmol) of 1-[3-(dimethylamino)propyl]-3-ethylcarbodiimide hydrochloride. After at least 12 hours at ambient temperature or until complete as determined by thin layer chromatography (95:5 dichloromethane/methanol eluent), the reaction mixture was washed sequentially with a saturated aqueous sodium bicarbonate solution and with distilled water. The organic layer was evaporated to give 1.41 g (quantitative yield) of the title compound as an off-white solid; ¹H NMR (CDCl₃) δ 1.39 (s, 9H); 2.61 (dd, J=6.5 Hz, J=17.2 Hz, 1H); 2.98 (dd, J=4.2 Hz, J=17.2 Hz, 1H); 4.41 (dd, J=5.9 Hz, J=15.3 Hz, 1H); 4.50-4.57 (m, 2H); 5.10 (s, 2H); 5.96-6.01 (m, 1H); 6.91-7.00 (m, 1H); 7.30-7.36 (m, 5H); 7.39-7.43 (m, 2H); 7.48-7.52 (m, 2H).

Examples 12-17 were prepared according to the procedure of Example 11, except that N-benzyloxycarbonyl-D-aspartic acid β-t-butyl ester monohydrate was replaced by the appropriate amino acid derivative, and 3-(trifluoromethyl)benzyl amine was replaced with the appropriate amine.

### Example 12

### N-Benzyloxycarbonyl-D-glutamic acid γ-t-butyl ester α-(3-trifluoromethyl)benzylamide

N-benzyloxycarbonyl-D-glutamic acid γ-t-butyl ester (1.14 g, 3.37 mmol) and 0.53 mL (3.70 mmol, Novabiochem) of 3-(trifluoromethyl)benzylamine gave 1.67 g (quantitative yield) of Example 12 as an off-white solid.

### Example 13

### N-Benzyloxycarbonyl-L-glutamic acid α-t-butyl ester γ-(4-cyclohexyl)piperazinamide

N-benzyloxycarbonyl-L-glutamic acid α-t-butyl ester (1.36 g, 4.03 mmol) and 0.746g (4.43 mmol) of 1-cyclohexylpiperazine gave 1.93 g (98%) of Example 13 as an off-white solid; ¹H NMR (CDCl₃) δ 1.02-1.12 (m, 5H); 1.43 (s, 9H), 1.60-1.64 (m, 1H); 1.80-1.93 (m, 5H); 2.18-2.52 (m, 8H); 3.38-3.60 (m, 41); 4.20-4.24 (m, 1H); 5.03-5.13 (m, 2H); 5.53-5.57 (m, 1H); 7.28-7.34 (m, 5H).

### Example 14

### N-Benzyloxycarbonyl-D-aspartic acid β-t-butyl ester α-(2-fluoro-3-trifluoromethyl)benzylamide

N-benzyloxycarbonyl-D-aspartic acid β-t-butyl ester monohydrate (Novabiochem) (0.25 g, 0.73 mmol) and 0.12 mL of (2-fluoro-3-trifluoromethyl)benzylamine gave 0.365 g (quantitative yield) of Example 14 as an off-white solid; ¹H NMR (CDCl₃) δ 1.38 (s, 9H); 2.59 (dd, J=6.5 Hz, J=17.0 Hz, 1H); 2.95 (dd, J=4.3 Hz, J=17.0 Hz, 1H); 4.46-4.56 (m, 3H); 5.11 (s, 2H); 5.94-5.96 (m, 1H); 7.15 (t, J=8.0 Hz, 1H); 7.30-7.36 (m, 5H); 7.47-7.52 (m, 2H).

### Example 15

### N-Benzyloxycarbonyl-D-aspartic acid β-t-butyl ester α-[(S)-α-methylbenzyl]amide

N-benzyloxycarbonyl-D-aspartic acid β-t-butyl ester monohydrate (Novabiochem) (0.25 g, 0.73 mmol) and 0.094 mL of (S)-α-methylbenzylamine gave 0.281 g (90%) of Example 15 as an off-white solid; ¹H NMR (CDCl₃) δ 1.41 (s, 9H); 1.44 (d, J=7.0 Hz, 3H); 2.61 (dd, J=7.0 Hz, J=17.0 Hz, 1H); 2.93 (dd, J=4.0 Hz, J=17.5 Hz, 1H); 4.50-4.54 (m, 1H); 5.04-5.14 (m, 3H); 5.94-5.96 (m, 1H); 6.76-6.80 (m, 1H); 7.21-7.37 (m, 10H).

### Example 16

### N-Benzyloxycarbonyl-D-aspartic acid β-t-butyl ester α-[(R)-α-methylbenzyl]amide

N-benzyloxycarbonyl-D-aspartic acid β-t-butyl ester monohydrate (Novabiochem) (0.25 g, 0.73 mmol) and 0.094 mL of (R)-α-methylbenzylamine gave 0.281 g (90%) of Example 16 as an off-white solid; ¹H NMR (CDCl₃) δ 1.38 (s, 9H); 1.43 (d, J=6.9 Hz, 3H); 2.54 (dd, J=7.3 Hz, J=17.2 Hz, 1H); 2.87 (dd, J=4.1 Hz, J=17.3 Hz, 1H); 4.46-4.50 (m, 1H); 4.99-5.15 (m, 3H); 5.92-5.96 (m, 1H); 6.78-6.82 (m, 1H); 7.21-7.33 (m, 10H).

### Example 17

### N-Benzyloxycarbonyl-D-aspartic acid γ-t-butyl ester α-[N-methyl-N-(3-trifluoromethylbenzyl)]amide

N-benzyloxycarbonyl-D-aspartic acid γ-t-butyl ester (0.303 g, 0.89 mmol, Novabiochem) and 0.168 g (0.89 mmol,) of N-methyl-N-(3-trifluoromethylbenzyl)amine gave 0.287 g (65%) of Example 17 as an off-white solid; ¹H NMR (CDCl₃) δ 1.40 (s, 9H); 2.55 (dd, J=5.8 Hz, J=15.8 Hz, 1H); 2.81 (dd, J=7.8 Hz, J=15.8 Hz, 1H); 3.10 (s, 3H); 4.25 (d, J=15.0 Hz, 1H); 4.80 (d, I=15.5 Hz, 1H); 5.01-5.13 (m, 3H); 5.52-5.55 (m, 1H); 7.25-7.52 (m, 10H).

### Example 18

### General Procedure for Hydrogenation of a Benzyloxycarbonyl Amine

L-aspartic acid β-t-butyl ester α-(3-trifluoromethyl)benzylamide.

A suspension of 2.23 g (4.64 mmol) of N-benzyloxycarbonyl-L-aspartic acid β-t-butyl ester α-(3-trifluoromethyl)benzylamide and palladium (5% wt. on activated carbon, 0.642 g) in 30 mL of methanol was held under an atmosphere of hydrogen until complete conversion as determined by thin layer chromatography (95:5 dichloromethane/methanol eluent). The reaction was filtered to remove the palladium over carbon and the filtrate was evaporated to give 1.52 g (96%) of the title compound as an oil; ¹H NMR (CDCl₃) δ 1.42 (s, 9H); 2.26 (brs, 2H); 2.63-2.71 (m, 1H); 2.82-2.87 (m, 1H), 3.75-3.77 (m, 1H); 4.47-4.50 (m, 2H); 7.41-7.52 (m, 4H); 7.90 (brs, 1H).

Examples 19-28 were prepared according to the procedure of Example 18, except that N-benzyloxycarbonyl-L-aspartic acid β-t-butyl ester α-(3-trifluoromethyl)benzylamide was replaced by the appropriate amino acid derivative.

### Example 19

### L-aspartic acid β-t-butyl ester α-[4-(2-phenylethyl)]piperazinamide

N-benzyloxycarbonyl-L-aspartic acid β-t-butyl ester α-[4-(2-phenylethyl)]piperazinamide (5.89 g, 11.9 mmol) gave 4.24 g (98%) of Example 19 as an off-white oil; ¹H NMR (CDCl₃): δ 1.42 (s, 9H); 2.61-2.95 (m, 10H); 3.60-3.90 (m, 4H); 4.35-4.45 (m, 1H); 7.17-7.29 (m, 5H).

### Example 20

### D-aspartic acid β-t-butyl ester α-(3-trifluoromethyl)benzylamide

N-benzyloxycarbonyl-D-aspartic acid β-t-butyl ester α-(3-trifluoromethyl)benzylamide (1.41 g, 2.93 mmol) gave 0.973 g (96%) of Example 20 as an off-white oil; ¹H NMR (CDCl₃): δ 1.42 (s, 9H); 2.21 (brs, 2H); 2.67 (dd, J=7.1 Hz, J=16.8 Hz, 1H); 2.84 (dd, J=3.6 Hz, J=16.7 Hz, 1H); 3.73-3.77 (m, 1H); 4.47-4.50 (m, 2H); 7.41-7.52 (m, 4H); 7.83-7.87 (m, 1H).

### Example 21

### L-glutamic acid γ-t-butyl ester α-(3-trifluoromethyl)benzylamide

N-benzyloxycarbonyl-L-glutamic acid γ-t-butyl ester α-(3-trifluoromethyl)benzylamide (5.41 g, 10.9 mmol) gave 3.94 g (quantitative yield) of Example 21 as an off-white oil; ¹H NMR (CDCl₃): δ 1.41 (s, 9H); 1.73-1.89 (m, 3H); 2.05-2.16 (m, 1H); 2.32-2.38 (m, 2H); 3.47 (dd, J=5.0 Hz, J=7.5 Hz, 1H); 4.47-4.49 (m, 2H); 7.36-7.54 (m, 4H); 7.69-7.77 (m, 1H).

### Example 22

### L-glutamic acid γ-t-butyl ester α-[4-(2-phenylethyl)]piperazinamide

N-benzyloxycarbonyl-L-glutamic acid γ-t-butyl ester α-[4-(2-phenylethyl)]piperazinamide (5.86 g, 11.50 mmol) gave 4.28 g (99%) of Example 22 as an off-white oil; ¹H NMR (CDCl₃) δ 1.39 (s, 9H); 2.00-2.08 (m, 1H); 2.38-2.46 (m, 1H); 2.55-2.90 (m, 9H); 3.61-3.82 (m, 4H); 4.48-4.56 (m, 1H); 7.17-7.26 (m, 5H).

### Example 23

### D-glutamic acid γ-t-butyl ester α-(3-trifluoromethyl)benzylamide

N-benzyloxycarbonyl-D-glutamic acid γ-t-butyl ester α-(3-trifluoromethyl)benzylamide (1.667 g, 3.37 mmol) gave 1.15 g (94%) of Example 23 as an off-white oil; ¹H NMR (CDCl₃) δ 1.41 (s, 9H); 1.80-2.20 (m, 4H); 2.31-2.40 (m, 2H); 3.51-3.59 (m, 1H); 4.47-4.49 (m, 2H); 7.39-7.52 (m, 4H); 7.71-7.79 (m, 1H).

### Example 24

### L-glutamic acid α-t-butyl ester γ-(4-cyclohexyl)piperazinamide

N-Benzyloxycarbonyl-L-glutamic acid α-t-butyl ester γ-(4-cyclohexyl)piperazinamide (1.93 g, 3.96 mmol) gave 1.30 g (93%) of Example 24 as an off-white oil; ¹H NMR (CDCl₃) δ 1.02-1.25 (m, 5H); 1.41 (s, 9H); 1.45-1.50 (m, 1H); 1.56-1.60 (m, 1H); 1.69-1.80 (m, 6H); 3.30 (dd, J=4.8 Hz, J=8.5 Hz, 1H); 3.44 (t, J=9.9 Hz, 2H); 3.56 (t, J=9.9 Hz, 2H).

### Example 25

### D-aspartic acid β-t-butyl ester α-(2-fluoro-3-trifluoromethyl)benzylamide

N-benzyloxycarbonyl-D-aspartic acid β-t-butyl ester α-(2-fluoro-3-trifluoromethyl)benzylamide (0.36 g, 0.72 mmol) gave 0.256 g (92%) of Example 25 as an off-white oil; ¹H NMR (CDCl₃) δ 1.39 (s, 9H); 2.50 (brs, 2H); 2.74 (dd, J=7.0 Hz, J=16.5 Hz, 1H); 2.86 (dd, J=4.8 Hz, J=16.8 Hz, 1H); 3.89 (brs, 2H); 4.47-4.57 (m, 2H); 7.16 (t, J=7.8 Hz, 1H); 7.48 (t, J=7.3 Hz, 1H); 7.56 (t, J=7.3 Hz, 1H); 7.97-8.02 (m, 1H).

### Example 26

### D-aspartic acid β-t-butyl ester α-[(S)-α-methylbenzyl]amide

N-benzyloxycarbonyl-D-aspartic acid β-t-butyl ester α-[(S)-α-methylbenzyl]amide (0.275 g, 0.65 mmol) gave 0.17 g (90%) of Example 26 as an off-white oil; ¹H NMR (CDCl₃) δ 1.40 (s, 9H); 1.47 (d, J=6.9 Hz, 3H); 1.98 (brs, 2H); 2.49 (dd, J=7.9 Hz, J=17.7 Hz, 1H); 2.83 (dd, J=3.6 Hz, J=16.7 Hz, 1H, 3.69 (brs, 1H); 4.99-5.10 (m, 1H); 7.19-7.33 (m, 5H); 7.65-7.68 (m, 1H).

### Example 27

### D-aspartic acid β-t-butyl ester α-[(R)-α-methylbenzyl]amide

N-benzyloxycarbonyl-D-aspartic acid β-t-butyl ester α-[(R)-α-methylbenzyl]amide (0.273 g, 0.64 mmol) gave 0.187 g (quantitative yield) of Example 27 as an off-white oil; ¹H NMR (CDCl₃) δ 1.38 (s, 9H); 1.46 (d, J=6.9 Hz, 3H); 1.79 (brs, 2H); 2.51 (dd, J=7.8 Hz, J=17.5 Hz, 1H); 2.87 (dd, J=3.6 Hz, J=16.9 Hz, 1H); 4.19 (brs, 1H); 4.99-5.11 (m, 1H); 7.18-7.34 (m, 5H); 7.86-7.90 (m, 1H).

### Example 28

### D-aspartic acid β-t-butyl ester α-[N-methyl-N-(3-trifluoromethylbenzyl)]amide

N-benzyloxycarbonyl-D-aspartic acid β-t-butyl ester α-[N-methyl-N-(3-trifluoromethylbenzyl)]amide (0.282 g, 0.57 mmol) gave 0.195 g (95%) of Example 28 as an off-white oil.

### Example 29

### General Procedure for Formation of a 2-azetidinone from an Imine and an Acetyl Chloride

Step 1: General procedure for formation of an imine from an amino acid derivative.

A solution of 1 equivalent of an α-amino acid ester or amide in dichloromethane is treated sequentially with 1 equivalent of an appropriate aldehyde, and a dessicating agent, such as magnesium sulfate or silica gel, in the amount of about 2 grams of dessicating agent per gram of starting α-amino acid ester or amide. The reaction is stirred at ambient temperature until all of the reactants are consumed as measured by thin layer chromatography (TLC). The reactions are typically complete within an hour. The reaction mixture is then filtered, the filter cake is washed with dichloromethane, and the filtrate concentrated under reduced pressure to provide the desired imine that is used as is in the subsequent step.

Step 2: General procedure for the 2+2 cycloaddition of an imine and an acetyl chloride.

A dichloromethane solution of the imine (10 mL dichloromethane/1 gram imine) is cooled to 0° C. To this cooled solution is added 1.5 equivalents of an appropriate amine, typically triethylamine, followed by the dropwise addition of a dichloromethane solution of 1.1 equivalents of an appropriate acetyl chloride, such as that described in Example 1 (10 mL dichloromethane/1 gm appropriate acetyl chloride). The reaction mixture is allowed to warm to ambient temperature over 1 h and is then quenched by the addition of a saturated aqueous solution of ammonium chloride. The resulting mixture is partitioned between water and dichloromethane. The layers are separated and the organic layer is washed successively with 1N hydrochloric acid, saturated aqueous sodium bicarbonate, and saturated aqueous sodium chloride. The organic layer is dried over magnesium sulfate and concentrated under reduced pressure. The residue may be used directly for further reactions, or purified by chromatography or by crystallization from an appropriate solvent system if desired.

### Example 30

### General Procedure for Hydrolysis of a tert-butyl Ester

A solution of tert-butyl ester derivative in formic acid, typically 1 g in 10 mL, is stirred at ambient temperature until no more ester is detected by thin layer chromatography (dichloromethane 95%/methanol 5%), a typical reaction time being around 3 hours. The formic acid is evaporated under reduced pressure; the resulting solid residue is partitioned between dichloromethane and saturated aqueous sodium bicarbonate. The organic layer is evaporated to give an off-white solid that may be used directly for further reactions, or recrystallized from an appropriate solvent system if desired.

### Example 31

### tert-Butyl 3(R)-[3(S)-(4(S)-phenyloxazolidin-2-on-3-yl)-3-methyl-4(R)-(styr-2-yl)azetidin-2-on-1-yl]-3-[(3-trifluoromethyl)phenylmethylaminocarbonyl]propanoate

Using the general method of Example 29, the imine prepared from 0.307 g (0.89 mmol) of D-aspartic acid β-t-butyl ester α-(3-trifluoromethyl)benzylamide (Example 20) and cinnamaldehyde was combined with 2-(4(S)-phenyloxazolidin-2-on-3-yl)propanoyl chloride (Example 4) to give 120 mg (20%) of the title compound after flash column chromatography purification (hexanes 70%/EtOAc 30%); ¹H NMR (CDCl₃) δ 1.25 (s, 3H), 1.38 (s, 9H); 3.09 (dd, J=3.0 Hz, J=18.0 Hz, 1H); 3.33 (dd, J=12.5 Hz, J=18.0 Hz, 1H); 4.01 (dd, J=3.0 Hz, J=11.5 Hz, 1H); 4.04 (dd, J=3.5 Hz, J=8.8 Hz, 1H); 4.42 (d, J=9.0 Hz, 1H); 4.45-4.51 (m, 3H); 4.61-4.66 (m, 1H); 4.75 (dd, J=3.5 Hz, J=8.5 Hz, 1H); 6.23 (dd, J=9.0 Hz, J=15.5 Hz, 1H); 6.78 (d, J=15.5 Hz, 1H); 7.23-7.53 (m, 13H); 7.64 (s, 1H).

### Example 32

### 3(R)-[3(S)-(4(S)-Phenyloxazolidin-2-on-3-yl)-3-methyl-4(R)-(styr-2-yl)azetidin-2-on-1-yl]-3-[(3-trifluoromethyl)phenylmethylaminocarbonyl]propanoic acid

Using the general method of Example 30, 120 mg (0.18 mmol) of Example 31 was hydrolyzed to give 108 mg (98%) of the title compound as an off-white solid; ¹H NMR (CDCl₃) δ 1.22 (s, 3H); 3.25 (dd, J=3.5 Hz, J=18.0 Hz, 1H); 3.36 (dd, J=10.8 Hz, J=18.2 Hz, 1H); 4.01 (dd, J=4.0 Hz, J=10.5 Hz, 1H); 4.05 (dd, J=3.8 Hz, J=8.8 Hz, 1H); 4.33 (d, J=9.0 Hz, 1H); 4.44-4.51 (m, 3H); 4.61-4.66 (m, 1H); 4.73 (dd, J=3.8 Hz, J=8.8 Hz, 1H); 6.19 (dd, J=9.0 Hz, J=16.0 Hz, 1H); 6.74 (d, J=16.0 Hz, 1H); 7.22-7.54 (m, 13H); 7.65 (s, 1H).

### Example 33

### 4-(Piperidin-1-yl)-piperidin-1-yl 3(R)-[3(S)-(4(S)-phenyloxazolidin-2-on-3-yl)-3-methyl-4(R)-(styr-2-yl)azetidin-2-on-1-yl]-3-[(3-trifluoromethyl)phenylmethylaminocarbonyl]propanoic acid

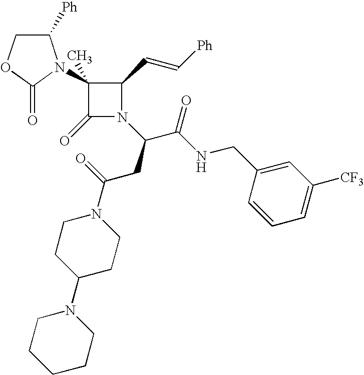

Using the procedure of Example 11, except that N-benzyloxycarbonyl-D-aspartic acid β-t-butyl ester monohydrate was replaced with the carboxylic acid of Example 33 and 3-(trifluoromethyl)benzyl amine was replaced with 4-(piperidin-1-yl)piperidine, the title compound was prepared in quantitative yield; MS (m+H)⁺ 772.

### Method Example 1

### Human Vasopression V₁ₐ Receptor Binding Assay

A cell line expressing the human V₁ₐ receptor in CHO cells (henceforth referred to as the hV₁ₐ cell line) was obtained from Dr. Michael Brownstein, NIMH, Bethesda, Md., USA. The hV₁ₐ cDNA sequence is described by Thibonnier et al., Journal of Biological Chemistry, 269:3304-3310 (1994), and the expression method was the same as described by Morel et al. (1992). The hV₁ₐ cell line was grown in alpha-MEM with 10% fetal bovine serum and 250 ug/ml G418 (Gibco, Grand Island, N.Y., USA). For competitive binding assay, hV1a cells were plated into 6-well culture plate at 1:10 dilution from a confluency flask, and maintained in culture for at least two days. Culture medium was then removed, cells were washed with 2 ml binding buffer (25 mM Hepes, 0.25% BSA, 1×DMEM, PH=7.0). To each well, 990 μl binding buffer containing 1 nM 3H-AVP was added, and followed by 10 μl series diluted Example compounds dissolved in DMSO. All incubations were in triplicate, and dose-inhibition curves consisted of total binding (DMSO) and 5 concentrations (0.1, 1.0, 10, 100, and 1000 nM) of test agents encompassing the IC₅₀. 100 nM cold AVP (Sigma) was used to assess non-specific binding. Cells were incubated for 45 minutes at 37° C., assay mixture was removed and each well was washed three times with PBS (pH=7.4). 1 ml 2% SDS was added per well and plates were let sit for 30 minutes. The whole content in a well was transferred to a scintillation vial. Each well was rinsed with 0.5 ml PBS which was then added to the corresponding vial. Scintillation fluid (Ecoscint, National Diagnostics, Atlanta, Ga.) was then added at 3 ml per vial. Samples were counted in a liquid scintillation counter (Beckman LS3801). IC₅₀ values were calculated by Prism Curve fitting software. Example 33 was tested according to Method Example 1, and exhibited an IC₅₀ in human V1ₐ of 5 nM.

### Method Example 2

### Inhibition of Phosphatidylinositol Turnover

The physiological effects of vasopressin are mediated through specific G-protein coupled receptors. The vasopressin V₁ₐ receptor is coupled to the G_{q}/G₁₁ family of G proteins and mediates phosphatidylinositol turnover. The agonist or antagonist character of the compounds of the invention may be determined by their ability to inhibit vasopressin-mediated turnover of phosphatidylinositol by the procedure described in the following paragraphs.

Cell Culture and Labeling of Cells.

Three days prior to the assay, near-confluent cultures of hV1a cells were dissociated and seeded in 6-well tissue culture plates, about 100 wells being seeded from each 75 cm² flask (equivalent to 12:1 split ratio). Each well contained 1 mL of growth medium with 2 μCi of [³H]myo-inositol (American Radiolabeled Chemicals, St. Louis, Mo., USA).

Incubations

All assays were in triplicate except for basal and 10 nM AVP (both n=6). AVP ((arginine vasopressin), Peninsula Labs, Belmont, Calif., USA (#8103)) was dissolved in 0.1N acetic acid. Test agents were dissolved in DMSO and diluted in DMSO to 200 times the final test concentration. Test agents and AVP (or corresponding volumes of DMSO) were added separately as 5 μL in DMSO to 12×75 mm glass tubes containing 1 mL of assay buffer (Tyrode's balanced salt solution containing 50 mM glucose, 10 mM LiCl, 15 mM HEPES pH 7.4, 10 μM phosphoramidon, and 100 μM bacitracin). The order of incubations was randomized. Incubations were initiated by removing the prelabeling medium, washing the monolayer once with 1 mL of 0.9% NaCl, and transferring the contents of the assay tubes to corresponding wells. The plates were incubated for 1 hour at 37° C. Incubations were terminated by removing the incubation medium and adding 500 μL of ice cold 5% (w/v) trichloroacetic acid and allowing the wells to stand for 15 min.

Measurement of [³H]inositol Phosphates

BioRad Poly-Prep Econo-Columns were packed with 0.3 mL of AG 1 X-8 100-200 formate form resin. Resin was mixed 1:1 with water and 0.6 mL added to each column. Columns were then washed with 10 mL water. Scintillation vials (20 mL) were placed under each column. For each well, the contents were transferred to a minicolumn, after which the well was washed with 0.5 mL distilled water, which was also added to the minicolumn. The columns were then washed twice with 5 mL of 5 mM myo-inositol to elute free inositol. Aliquots (1 mL) were transferred to 20 mL scintillation vials and 10 mL of Beckman Ready Protein Plus added. After the myo-inositol wash was complete, empty scintillation vials were placed under the columns, and [³H]inositol phosphates were eluted with three additions of 1 mL 0.5 M ammonium formate containing 0.1 N formic acid. Elution conditions were optimized to recover inositol mono-, bis-, and trisphosphates, without eluting the more metabolically inert tetrakis-, pentakis-, and hexakis-phosphates. To each sample was added 10 mL of a high salt capacity scintillation fluid such as Tru-Count High Salt Capacity or Packard Hionic-Fluor. Inositol lipids were measured by adding 1 mL of 2% sodium dodecyl sulfate (SDS) to each well, allowing the wells to stand for at least 30 min., and transferring the solution to 20 mL scintillation vials, to which 10 mL Beckman Ready Protein Plus scintillation fluid was then added. Samples were counted in a Beckman LS 3801 liquid scintillation counter for 10 min. Total inositol incorporation for each well was calculated as the sum of free inositol, inositol phosphates, and inositol lipids.

Data Analysis: Concentration-Inhibition Experiments

Concentration-response curves for AVP and concentration-inhibition curves for test agents versus 10 nM AVP were analyzed by nonlinear least-squares curve-fitting to a 4-parameter logistic function. Parameters for basal and maximal inositol phosphates, EC₅₀ or IC₅₀, and Hill coefficient were varied to achieve the best fit. The curve-fitting was weighted under the assumption that the standard deviation was proportional to dpm of radioactivity. Full concentration-response curves for AVP were run in each experiment, and IC₅₀ values were converted to Kᵢ values by application of the Cheng-Prusoff equation, based on the EC₅₀ for AVP in the same experiment. Inositol phosphates were expressed as dpm per 10⁶ dpm of total inositol incorporation.

Data Analysis: Competitivity Experiments

Experiments to test for competitivity of test agents consisted of concentration-response curves for AVP in the absence and presence of two or more concentrations of test agent. Data were fit to the following competitive logistic equation:

$$Y = B + \frac{M \times {\{ A / [ E + ( D / K ) ] \}}^{Q}}{1 + {\{ A / [ E + ( D / K ) ] \}}^{Q}}$$

where Y is dpm of inositol phosphates, B is concentration of basal inositol phosphates, M is the maximal increase in concentration of inositol phosphates, A is the concentration of agonist (AVP), E is the EC₅₀ for agonist, D is the concentration of the antagonist, K is the Kᵢ for antagonist, and Q is the cooperativity (Hill coefficient).

Vasopressin V₁ₐ receptors are also known to mediate platelet aggregation. Vasopressin V₁ₐ receptor agonists cause platelet aggregation, while vasopressin V₁ₐ receptor antagonists inhibit the platelet aggregation precipitated by vasopressin or vasopressin V₁ₐ agonists. The degree of antagonist activity of the compounds of the invention may be determined by the assay described in the following paragraphs.

Blood from healthy, human volunteers was collected by venipuncture and mixed with heparin (60 mL of blood added to 0.4 mL of heparanized saline solution (4 mg heparin/mL saline)). Platelet-rich plasma (PRP) was prepared by centrifuging whole blood (150×g), and indomethacin (3 μM) was added to PRP to block the thromboxane-mediated release reaction. PRP was continuously stirred at 37° C. and change in optical density was followed after the addition of arginine vasopressin (AVP) (30 nM) to initiate aggregation. Compounds were dissolved in 50% dimethylsulfoxide (DMSO) and added (10 μL/415 μL PRP) before the addition of AVP. The percent inhibition of AVP-induced aggregation was measured and an IC₅₀ calculated.

In studies using washed platelets, 50 mL of whole blood was mixed with 10 mL of citrate/heparin solution (85 mM sodium citrate, 64 mM citric acid, 111 mM glucose, 5 units/mL heparin) and PRP isolated as described above. PRP was then centrifuged (150×g) and the pellet resuspended in a physiologic buffer solution (10 mM HEPES, 135 mM sodium chloride, 5 mM potassium chloride, and 1 mM magnesium chloride) containing 10 μM indomethicin. Human fibrinogen (0.2 mg/mL) and calcium chloride (1 mM) were added to stirred platelets before initiating aggregation with AVP (30 nM) as previously described.

The activity of compounds of formulae I, II, and III in the antagonism of the vasopressin V₁ₐ receptor provides a method of antagonizing the vasopressin V₁ₐ receptor comprising administering to a subject in need of such treatment an effective amount of a compound of that formula. It is known that numerous physiological and therapeutic benefits are obtained through the administration of drugs that antagonize the vasopressin V₁ₐ receptor. These activities may be catagorized as peripheral and central. Peripheral utilities include administration of vasopressin V₁ₐ antagonists of formula I as adjuncts in heart failure or as antithrombotic agents. Central effects include administration of vasopressin V₁ₐ antagonists of formulae I, II, and III in the treatment of obsessive-compulsive disorder, aggressive disorders, depression and anxiety.

Obsessive-compulsive disease appears in a great variety of degrees and symptoms, generally linked by the victim's uncontrollable urge to perform needless, ritualistic acts. Acts of acquiring, ordering, cleansing and the like, beyond any rational need or rationale, are the outward characteristic of the disease. A badly afflicted subject may be unable to do anything but carry out the rituals required by the disease. Obsessive-compulsive disease, in all its variations, is a preferred target of treatment with the present adjunctive therapy method and compositions. The utility of the compounds of formulae I, II, and III in the treatment of obsessive-compulsive disorder was demonstrated as described in the following assay.

In golden hamsters, a particular stereotypy, flank marking behavior, can be induced by microinjections of vasopressin (10-100 nL, 1-100 μM) into the anterior hypothalamus (Ferris et al., Science, 224:521-523 (1984); Albers and Ferris, Regulatory Peptides, 12:257-260 (1985); Ferris et al., European Journal of Pharmacology, 154:153-159 (1988)). Following the releasing stimulus, the behavior is initiated by grooming, licking and combing of the large sebaceous glands on the dorsolateral flanks. Bouts of flank gland grooming may be so intense that the flank region is left matted and soaked in saliva. After grooming, the hamsters display flank marking behavior, a type of scent marking involved in olfactory communication (Johnston, Physio. Behav., 51:437-448 (1985); Ferris et al., Physio. Behav., 40:661-664 (1987)), by arching the back and rubbing the flank glands vigorously against any vertical surface. Vasopressin-induced flank marking is usually induced within a minute after the microinjection (Ferris et al., Science, 224:521-523 (1984)). The behavior is specific to vasopressin, as micro-injections of other neuropeptides, excitatory amino acids, and catecholamines do not elicit flank marking (Ferris et al., Science, 224:521-523 (1984); Albers and Ferris, Regulatory Peptides, 12:257-260 (1985)). Furthermore, flank marking is specific to the vasopressin V₁ receptor, as the behavior is selectively inhibited by V₁ receptor antagonists and activated by V₁ receptor agonists (Ferris et al., Neuroscience Letters, 55:239-243 (1985); Albers et al., Journal of Neuroscience, 6:2085-2089 (1986); Ferris et al., European Journal of Pharmacology, 154:153-159 (1988)).

All animals were adult male golden hamsters (Mesocricetus auratus) weighing approximately 160 gm. The animals underwent stereotaxic surgery, and were allowed to recover before behavioral testing. The hamsters were kept on a reverse light cycle (14 hr light, 10 hr dark, lights on at 19:00) in Plexiglas™ cages, and received food and water ad libitum.

Stereotaxic surgery was performed under pentobarbital anesthesia. The stereotaxic coordinates were: 1.1 mm anterior to the bregma, 1.8 mm lateral to the midsagittal suture at an 8° angle from the verticle line, and 4.5 mm below the dura. The nose bar was placed at the level of the interaural line. An unilateral 26-gauge guide cannula was lowered to the site and secured to the skull with dental cement. The guide cannulae were closed with a 33-gauge obturator extending 1 mm beyond the guide. The innercanulae used for the microinjections extended 3.0 mm beyond the guide to reach the anterior hypothalamus.

The hamsters were microinjected with 1 μM vasopressin in a volume of 150 nL. The vasopressin was given as a cocktail with 200 mM, 20 mM, 2 mM of the test compound or alone, in the vehicle, dimethylsulfoxide. Both the vasopressin and the test compound were dissolved in 100% dimethylsulfoxide. All injections were aimed at the anterior hypothalamus. Animals were scored for flank marking for a period of 10 minutes in a clean cage.

Another aspect of this invention is the use of compounds of formulae I, II, and III in combination with a serotonin reuptake inhibitor for use in the treatment of obsessive-compulsive disease, aggressive disorder, or depression. Compounds useful as serotonin reuptake inhibitors include but are not limited to:

Fluoxetine, N-methyl-3-(p-trifluoromethylphenoxy)-3-phenylpropylamine, is marketed in the hydrochloride salt form, and as the racemic mixture of its two enantiomers. U.S. Pat. No. 4,314,081 is an early reference on the compound. Robertson et al., J. Med. Chem., 31:1412 (1988), taught the separation of the R and S enantiomers of fluoxetine and showed that their activity as serotonin uptake inhibitors is similar to each other. In this document, the word “fluoxetine” will be used to mean any acid addition salt or the free base, and to include either the racemic mixture or either of the R and S enantiomers;

Duloxetine, N-methyl-3-(1-naphthalenyloxy)-3-(2-thienyl)propanamine, is usually administered as the hydrochloride salt and as the (+) enantiomer. It was first taught by U.S. Pat. No. 4,956,388, which shows its high potency. The word “duloxetine” will be used here to refer to any acid addition salt or the free base of the molecule;

Venlafaxine is known in the literature, and its method of synthesis and its activity as an inhibitor of serotonin and norepinephrine uptake are taught by U.S. Pat. No. 4,761,501. Venlafaxine is identified as compound A in that patent;

Milnacipran (N,N-diethyl-2-aminomethyl-1-phenylcyclopropanecarboxamide) is taught by U.S. Pat. No. 4,478,836, which prepared milnacipran as its Example 4. The patent describes its compounds as antidepressants. Moret et al., Neuropharmacology, 24:1211-19 (1985), describe its pharmacological activities as an inhibitor of serotonin and norepinephrine reuptake;

Citalopram, 1-[3-(dimethylamino)propyl]-1-(4-fluorophenyl)-1,3-dihydro-5-isobenzofurancarbonitrile, is disclosed in U.S. Pat. No. 4,136,193 as a serotonin reuptake inhibitor. Its pharmacology was disclosed by Christensen et al., Eur. J. Pharmacol., 41:153 (1977), and reports of its clinical effectiveness in depression may be found in Dufour et al., Int. Clin. Psychopharmacol., 2:225 (1987), and Timmerman et al., ibid., 239;

Fluvoxamine, 5-methoxy-1-[4-(trifluoromethyl)phenyl]-1-pentanone O-(2-aminoethyl)oxime, is taught by U.S. Pat. No. 4,085,225. Scientific articles about the drug have been published by Claassen et al., Brit. J. Pharmacol., 60:505 (1977); and De Wilde et al., J. Affective Disord., 4:249 (1982); and Benfield et al., Drugs, 32:313 (1986);

Paroxetine, trans-(−)-3-[(1,3-benzodioxol-5-yloxy)methyl]-4-(4-fluorophenyl)piperidine, may be found in U.S. Pat. Nos. 3,912,743 and 4,007,196. Reports of the drug's activity are in Lassen, Eur. J. Pharmacol., 47:351 (1978); Hassan et al., Brit. J. Clin. Pharmacol., 19:705 (1985); Laursen et al., Acta Psychiat. Scand., 71:249 (1985); and Battegay et al., Neuropsychobiology, 13:31 (1985); and

Sertraline, (1S-cis)-4-(3,4-dichlorophenyl)-1,2,3,4-tetrahydro-N-methyl-1-naphthylamine hydrochloride, a serotonin reuptake inhibitor disclosed in U.S. Pat. No. 4,536,518, is marketed as an antidepressant.

All of the above-referenced patents are hereby incorporated by reference.

The adjunctive therapy of this aspect of the present invention is carried out by administering a vasopressin V₁ₐ antagonist together with a serotonin reuptake inhibitor in any manner that provides effective levels of the compounds in the body at the same time. All of the compounds concerned are orally available and are normally administered orally, and so oral administration of the adjunctive combination is preferred. They may be administered together, in a single dosage form, or may be administered separately.

This aspect of the present invention provides a potentiation of the decrease in the concentration of vasopressin observed as an effect of administration of a vasopressin V₁ₐ antagonist by administration of a serotonin reuptake inhibitor. This aspect of the present invention is particularly suited for use in the treatment of depression and obsessive compulsive disorder. Such disorders may often be resistant to treatment with a serotonin reuptake inhibitor alone.

### Method Example 3

### Human Oxytocin Binding and Functional Assay

Compounds of the present invention are believed to be oxytocin agents. Oxytocin preparations and a number of oxytocin agonists are commercially available for therapeutic use. In recent years, oxytocin antagonists with antiuterotonic activity have been developed and evaluated for their potential use in the treatment of preterm labor and dysmenorrhyea (Pavo et al., J. Med. Chem., 37:255-259 (1994); Akerlund et al., Br. J. Obstet. Gynaecol., 94:1040-1044 (1987); Akerlund et al., Br. J. Obstet. Gynaecol., 86:484-487 (1979)). The oxytocin antagonist atosiban has been studied clinically and resulted in a more significant inhibition of preterm contractions than did placebo (Goodwin et al., Am. J. Obstet. Gynecol., 170:474 (1994)).

The human oxytocin receptor has been cloned and expressed (Kimura et al., Nature, 356:526-529 (1992)), it is identified under the accession number X64878. To demonstrate the affinity of the compounds of the present invention for the human oxytocin receptor, binding studies were performed using a cell line expressing the human oxytocin receptor in 293 cells (henceforth referred to as the OTR cell line) substantially by the procedure described by Morel et al. (Nature, 356:523-526 (1992)). The 293 cell line is a permanent line of primary human embryonal kidney cells transformed by sheared human adenovirus type 5 DNA. It is identified as ATCC CRL-1533.

The OTR cell line was grown in DMEM (Delbecco's Modified Essential Medium, Sigma, St. Louis, Mo., USA) with 10% fetal bovine serum, 2 mM L-glutamine, 200 μg hygromycin (Sigma, St. Louis, Mo., USA) and 250 μg/ml G418 (Gibco, Grand Island, N.Y., USA). To prepare membranes, OTR cells were grown to confluency in 20 roller bottles. Cells were dissociated with enzyme-free cell dissociation medium (Specialty Media, Lavallette, N.J., USA) and centrifuged at 3200 rpm for 15 minutes. The pellet was resuspended in 40 mL of Tris-HCl (tris[hydroxymethyl]aminomethane hydrochloride) buffer (50 mM, pH 7.4) and homogenized for 1 minute with a Tekmar Tissumizer (Cincinnatti, Ohio USA). The suspension was centrifuged at 40,000×g for 10 minutes. The pellet was resuspended and centrifuged as above. The final pellet was suspended in 80 mL of Tris 7.4 buffer and stored in 4 mL aliquots at −80° C. For assay, aliquots were resuspended in assay buffer and diluted to 375 μg protein per mL. Protein concentration was determined by BCA assay (Pierce, Rockford, Ill., USA).

Assay buffer was 50 mM Tris-HCl (tris[hydroxymethyl]aminomethane hydrochloride), 5 mM MgCl₂, and 0.1% bovine serum albumin at pH 7.4. The radioligand for binding assays was [³H]oxytocin ([tyrosyl-2,6-³H]oxytocin, 48.5 Ci/mmol, DuPont NEN, Boston, Mass., USA). The order of additions was 195 μL assay buffer, 200 μL OTR membranes (75 μg protein) in assay buffer, 5 μL of test agent in dimethylsulfoxide (DMSO) or DMSO alone, and 100 μL [³H]oxytocin in assay buffer (final concentration 1.0 nM). Incubations were for one hour at room temperature. Bound radioligand was separated from free by filtration on a Brandel cell harvester (Gaithersburg, Md., USA) through Whatman GF/B glass-fiber filters that had been soaked for 2 hours in 0.3% polyethylenimine. The filters were washed with ice-cold 50 mM Tris-HCl (pH 7.7 at 25° C.) and the filter circles were placed in scintillation vials, to which were then added 5 mL. Ready Protein Plus™ scintillation fluid, and counted in a liquid scintillation counter. All incubations were in triplicate, and dose-inhibition curves consisted of total binding, nonspecific binding (100 μM oxytocin, Sigma, St. Louis, Mo., USA), and 6 or 7 concentrations of test agent encompassing the IC₅₀. Total binding was typically about 1,000 cpm and nonspecific binding about 200 cpm. IC₅₀ values were calculated by nonlinear least-squares curve-fitting to a 4-parameter logistic model. Certain compounds of formula I have shown affinity for the oxytocin receptor.

Several bioassays are available to determine the agonist or antagonist character of compounds exhibiting affinity at the oxytocin receptor. One such assay is described in U.S. Pat. No. 5,373,089, hereby incorporated by reference. Said bioassay is derived from procedures described in a paper by Sawyer et al. (Endocrinology, 106:81 (1980)), which in turn was based on a report of Holton (Brit. J. Pharmacol., 3:328 (1948)). The assay calculations for pA₂ estimates are described by Schild (Brit. J. Pharmacol., 2:189 (1947)).

Assay Method

1. Animals: a 1.5 cm piece of uterus from a virgin rat (Holtzman) in natural estrus is used for the assay.

2. Buffer/Assay Bath: The buffer used is Munsicks. This buffer contains 0.5 mM Mg²⁺. The buffer is gassed continuously with 95% oxygen/5% carbon dioxide giving a pH of 7.4. The temperature of the assay bath is 37° C. A 10 mL assay bath is used that contains a water jacket for maintaining the temperature and inlet and outlet spikets for adding and removing buffer.

3. Polygraph/transducer: The piece of uterine tissue used for the assay is anchored at one end and connected to a Statham Strain Gauge Force Transducer at the other end which in turn is attached to a Grass Polygraph Model 79 for monitoring the contractions.

4. Assay Protocol:

(a) The tissue is equilibrated in the assay bath for one hour with washing with new buffer every 15 minutes. One gram of tension is kept on the tissue at all times.

(b) The tissue is stimulated initially with oxytocin at 10 nM to acclimate the tissue and with 4 mM potassium chloride (KCl) to determine the maximum contractile response.

(c) A cumulative dose response curve is then done with oxytocin and a concentration of oxytocin equivalent to approximately 80% of the maximum is used for estimating the pA₂ of the antagonist.

(d) The tissue is exposed to oxytocin (Calbiochemical, San Diego, Calif.) for one minute and washed out. There is a three minute interval before addition of the next dose of agonist or antagonist. When the antagonist is tested, it is given five minutes before the agonist. The agonist is given for one minute. All responses are integrated using a 7P10 Grass Integrator. A single concentration of oxytocin, equal to 80% of the maximum response, is used to test the antagonist. Three different concentrations of antagonists are used, two that will reduce the response to the agonist by less than 50% and one that will reduce the response greater than 50% (ideally this relation would be 25%, 50% and 75%). This is repeated three times for each dose of antagonist for a three point assay.

(e) Calculations for pA₂-The dose-response (DR) ratios are calculated for antagonist and a Schild's Plot is performed by plotting the Log (DR-1) vs. Log of antagonist concentration. The line plotted is calculated by least-squares regression analysis. The pA₂ is the concentration of antagonist at the point where the regression line crosses the 0 point of the Log (DR-1) ordinate. The pA₂ is the negative Log of the concentration of antagonist that will reduce the response to the agonist by one-half.

Oxytocin is known for its hormonal role in parturition and lactation. Oxytocin agonists are useful clinically to induce lactation; induce or augment labor, control postpartum uterine atony and hemmorhage; cause uterine contraction after cesarean section or during other uterine surgery; and to induce therapeutic abortion. Oxytocin, acting as a neurotransmitter in the central nervous system, also plays an important role in the expression of central functions such as maternal behavior, sexual behavior (including penile erection, lordosis and copulatory behavior), yawning, tolerance and dependance mechanisms, feeding, grooming, cardiovascular regulation and thermoregulation (Argiolas and Gessa, Neuroscience and Biobehavioral Reviews, 15:217-231 (1991)). Oxytocin antagonists find therapeutic utility as agents for the delay or prevention of premature labor, or to slow or arrest delivery for brief periods in order to undertake other therapeutic measures.

### Method Example 4

### Tachykinin Receptor Binding Assay

Compounds of the present invention are believed to be tachykinin agents. Tachykinins are a family of peptides which share a common amidated carboxy terminal sequence. Substance P was the first peptide of this family to be isolated, although its purification and the determination of its primary sequence did not occur until the early 1970's. Between 1983 and 1984 several groups reported the isolation of two novel mammalian tachykinins, now termed neurokinin A (also known as substance K, neuromedin 1, and neurokinin α), and neurokinin B (also known as neuromedin K and neurokinin β). See, J. E. Maggio, Peptides, 6 (Supplement 3): 237-243 (1985) for a review of these discoveries.

Tachykinins are widely distributed in both the central and peripheral nervous systems. When released from nerves, they exert a variety of biological actions, which, in most cases, depend upon activation of specific receptors expressed on the membrane of target cells. Tachykinins are also produced by a number of non-neural tissues. The mammalian tachykinins substance P, neurokinin A, and neurokinin B act through three major receptor subtypes, denoted as NK-1, NK-2, and NK-3, respectively. These receptors are present in a variety of organs.

Substance P is believed inter alia to be involved in the neurotransmission of pain sensations, including the pain associated with migraine headaches and with arthritis. These peptides have also been implicated in gastrointestinal disorders and diseases of the gastrointestinal tract such as inflammatory bowel disease. Tachykinins have also been implicated as playing a role in numerous other maladies, as discussed infra.

In view of the wide number of clinical maladies associated with an excess of tachykinins, the development of tachykinin receptor antagonists will serve to control these clinical conditions. The earliest tachykinin receptor antagonists were peptide derivatives. These antagonists proved to be of limited pharmaceutical utility because of their metabolic instability. Recent publications have described novel classes of non-peptidyl tachykinin receptor antagonists which generally have greater oral bioavailability and metabolic stability than the earlier classes of tachykinin receptor antagonists. Examples of such newer non-peptidyl tachykinin receptor antagonists are found in European Patent Publication 591,040 A1, published Apr. 6, 1994; Patent Cooperation Treaty publication WO 94/01402, published Jan. 20, 1994; Patent Cooperation Treaty publication WO 94/04494, published Mar. 3, 1994; Patent Cooperation Treaty publication WO 93/011609, published Jan. 21, 1993, Patent Cooperation Treaty publication WO 94/26735, published Nov. 24, 1994. Assays useful for evaluating tachykinin receptor antagonists are well known in the art. See, e.g., J. Jukic et al, Life Sciences, 49:1463-1469 (1991); N. Kucharczyk et al., Journal of Medicinal Chemistry, 36:1654-1661 (1993); N. Rouissi et al., Biochemical and Biophysical Research Communications, 176:894-901 (1991).

### Method Example 5

### NK-1 Receptor Binding Assay

Radioreceptor binding assays were performed using a derivative of a previously published protocol. D. G. Payan et al., Journal of Immunology, 133:3260-3265 (1984). In this assay an aliquot of IM9 cells (1×10⁶ cells/tube in RPMI 1604 medium supplemented with 10% fetal calf serum) was incubated with 20 pM ¹²⁵I-labeled substance P in the presence of increasing competitor concentrations for 45 minutes at 4° C.

The IM9 cell line is a well-characterized cell line which is readily available to the public. See, e.g., Annals of the New York Academy of Science, 190:221-234 (1972); Nature (London), 251:443-444 (1974); Proceedings of the National Academy of Sciences (USA), 71:84-88 (1974). These cells were routinely cultured in RPMI 1640 supplemented with 50 μg/mL gentamicin sulfate and 10% fetal calf serum.

The reaction was terminated by filtration through a glass fiber filter harvesting system using filters previously soaked for 20 minutes in 0.1% polyethylenimine. Specific binding of labeled substance P was determined in the presence of 20 nM unlabeled ligand.

### Method Example 6

### NK-2 Receptor Binding Assay

The CHO-hNK-2R cells, a CHO-derived cell line transformed with the human NK-2 receptor, expressing about 400,000 such receptors per cell, were grown in 75 cm² flasks or roller bottles in minimal essential medium (alpha modification) with 10% fetal bovine serum. The gene sequence of the human NK-2 receptor is given in N. P. Gerard et al., Journal of Biological Chemistry, 265:20455-20462 (1990).

For preparation of membranes, 30 confluent roller bottle cultures were dissociated by washing each roller bottle with 10 ml of Dulbecco's phosphate buffered saline (PBS) without calcium and magnesium, followed by addition of 10 ml of enzyme-free cell dissociation solution (PBS-based, from Specialty Media, Inc.). After an additional 15 minutes, the dissociated cells were pooled and centrifuged at 1,000 RPM for 10 minutes in a clinical centrifuge. Membranes were prepared by homogenization of the cell pellets in 300 mL 50 mM Tris buffer, pH 7.4 with a Tekmar® homogenizer for 10-15 seconds, followed by centrifugation at 12,000 RPM (20,000×g) for 30 minutes using a Beckman JA-14® rotor. The pellets were washed once using the above procedure. and the final pellets were resuspended in 100-120 mL 50 mM Tris buffer, pH 7.4, and 4 ml aliquots stored frozen at −70° C. The protein concentration of this preparation was 2 mg/mL.

For the receptor binding assay, one 4-mL aliquot of the CHO-hNK-2R membrane preparation was suspended in 40 mL of assay buffer containing 50 mM Tris, pH 7.4, 3 mM manganese chloride, 0.02% bovine serum albumin (BSA) and 4 μg/mL chymostatin. A 200 μL volume of the homogenate (40 μg protein) was used per sample. The radioactive ligand was [¹²⁵I]iodohistidyl-neurokinin A (New England Nuclear, NEX-252), 2200 Ci/mmol. The ligand was prepared in assay buffer at 20 nCi per 100 μL; the final concentration in the assay was 20 pM. Non-specific binding was determined using 1 μM eledoisin. Ten concentrations of eledoisin from 0.1 to 1000 nM were used for a standard concentration-response curve.

All samples and standards were added to the incubation in 10 μL dimethylsulfoxide (DMSO) for screening (single dose) or in 5 μL DMSO for IC₅₀ determinations. The order of additions for incubation was 190 or 195 μL assay buffer, 200 μL homogenate, 10 or 5 μL sample in DMSO, 100 μL radioactive ligand. The samples were incubated 1 hr at room temperature and then filtered on a cell harvester through filters which had been presoaked for two hours in 50 mM Tris buffer, pH 7.7, containing 0.5% BSA. The filter was washed 3 times with approximately 3 mL of cold 50 mM Tris buffer, pH 7.7. The filter circles were then punched into 12×75 mm polystyrene tubes and counted in a gamma counter.

Tachykinin receptor antagonists are of value in the treatment of a wide variety of clinical conditions which are characterized by the presence of an excess of tachykinin. These clinical conditions may include disorders of the central nervous system such as anxiety, depression, psychosis, and schizophrenia; neurodegenerative disorders such as dementia, including senile dementia of the Alzheimer's type, Alzheimer's disease, AIDS-associated dementia, and Down's syndrome; demyelinating diseases such as multiple sclerosis and amyotrophic lateral sclerosis and other neuropathological disorders such as peripheral neuropathy, such as diabetic and chemotherapy-induced neuropathy, and post-herpetic and other neuralgias; acute and chronic obstructive airway diseases such as adult respiratory distress syndrome, bronchopneumonia, bronchospasm, chronic bronchitis, drivercough, and asthma; inflammatory diseases such as inflammatory bowel disease, psoriasis, fibrositis, osteoarthritis, and rheumatoid arthritis; disorders of the musculo-skeletal system, such as osteoporosis; allergies such as eczema and rhinitis; hypersensitivity disorders such as poison ivy; ophthalmic diseases such as conjunctivitis, vernal conjunctivitis, and the like; cutaneous diseases such as contact dermatitis, atopic dermatitis, urticaria, and other eczematoid dermatites; addiction disorders such as alcoholism; stress-related somatic disorders; reflex sympathetic dystrophy such as shoulder/hand syndrome; dysthymic disorders; adverse immunological reactions such as rejection of transplanted tissues and disorders related to immune enhancement or suppression such as systemic lupus erythematosis; gastrointestinal disorders or diseases associated with the neuronal control of viscera such as ulcerative colitis, Crohn's disease, emesis, and irritable bowel syndrome; disorders of bladder function such as bladder detrusor hyper-reflexia and incontinence; artherosclerosis; fibrosing and collagen diseases such as scleroderma and eosinophilic fascioliasis; irritative symptoms of benign prostatic hypertrophy, disorders of blood flow caused by vasodilation and vasospastic diseases such as angina, migraine, and Raynaud's disease; and pain or nociception, for example, that attributable to or associated with any of the foregoing conditions, especially the transmission of pain in migraine.

NK-1 antagonists are useful in the treatment of pain, especially chronic pain, such as neuropathic pain, post-operative pain, and migraines, pain associated with arthritis, cancer-associated pain, chronic lower back pain, cluster headaches, herpes neuralgia, phantom limb pain, central pain, dental pain, neuropathic pain, opioid-resistant pain, visceral pain, surgical pain, bone injury pain, pain during labor and delivery, pain resulting from burns, including sunburn, post partum pain, angina pain, and genitourinary tract-related pain including cystitis.

In addition to pain, NK-1 antagonists are especially useful in the treatment and prevention of urinary incontinence; irritative symptoms of benign prostatic hypertrophy, motility disorders of the gastrointestinal tract, such as irritable bowel syndrome; acute and chronic obstructive airway diseases, such as bronchospasm, bronchopneumonia, asthma, and adult respiratory distress syndrome; artherosclerosis; inflammatory conditions, such as inflammatory bowel disease, ulcerative colitis, Crohn's disease, rheumatoid arthritis, osteoarthritis, neurogenic inflammation, allergies, rhinitis, cough, dermatitis, urticaria, psoriasis, conjunctivitis, emesis, irritation-induced miosis; tissue transplant rejection; plasma extravasation resulting from cytokine chemotherapy and the like; spinal cord trauma; stroke; cerebral stroke (ischemia); Alzheimer's disease; Parkinson's disease; multiple sclerosis; amyotrophic lateral sclerosis; schizophrenia; anxiety, and depression.

NK-2 antagonists are useful in the treatment of urinary incontinence, bronchospasm, asthma, adult respiratory distress syndrome, motility disorders of the gastrointestinal tract, such as irritable bowel syndrome, and pain.

In addition to the above indications the compounds of the invention may be useful in the treatment of emesis, including acute, delayed, or anticipatory emesis, such as emesis induced by chemotherapy, radiation, toxins, pregnancy, vestibular disorders, motion, surgery, migraine, and variations in intercranial pressure. Most especially, the compounds of formulae I, II, and III are of use in the treatment of emesis induced by antineoplastic (cytotoxic) agents including those routinely used in cancer chemotherapy.

Examples of such chemotherapeutic agents include alkylating agents, for example, nitrogen mustards, ethyleneimine compounds, alkyl sulfonates, and other compounds with an alkylating action, such as nitrosoureas, cisplatin, and dacarbazine; antimetabolites, for example, folic acid, purine, or pyrimidine antagonists; mitotic inhibitors, for example vinca alkaloids and derivatives of podophyllotoxin; and cytotoxic antibiotics.

Particular examples of chemotherapeutic agents are described, for instance, by D. J. Stewart in NAUSEA AND VOMTING: RECENT RESEARCH AND CLINICAL ADVANCES, (J. Kucharczyk et al, eds., 1991), at pages 177-203. Commonly used chemotherapeutic agents include cisplatin, dacarbazine (DTIC), dactinomycin, mechlorethamine (nitrogen mustard), streptozocin, cyclophosphamide, carmustine (BCNU), lomustine (CCNU), doxorubicin, daunorubicin, procarbazine, mitomycin, cytarabine, etoposide, methotrexate, 5-fluorouracil, vinblastine, vincristine, bleomycin, and chlorambucil. R. J. Gralla et al., Cancer Treatment Reports, 68:163-172 (1984).

The compounds of formulae I, II, and III may also be of use in the treatment of emesis induced by radiation, including radiation therapy such as in the treatment of cancer, or radiation sickness; and in the treatment of post-operaive nausea and vomiting.

While it is possible to administer a compound employed in the methods of this invention directly without any formulation, the compounds are usually administered in the form of pharmaceutical compositions comprising a pharmaceutically acceptable excipient and at least one active ingredient. These compositions can be administered by a variety of routes including oral, rectal, transdermal, subcutaneous, intravenous, intramuscular, and intranasal. Many of the compounds employed in the methods of this invention are effective as both injectable and oral compositions. Such compositions are prepared in a manner well known in the pharmaceutical art and comprise at least one active compound. See, e.g., REMINGTON'S PHARMACEUTICAL SCIENCES, (16th ed. 1980).

In making the compositions employed in the present invention the active ingredient is usually mixed with an excipient, diluted by an excipient, or enclosed within such a carrier which can be in the form of a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing for example up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form, each dosage containing from about 0.05 to about 100 mg, more usually about 1.0 to about 30 mg, of the active ingredient. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient.

The active compounds are generally effective over a wide dosage range. For examples, dosages per day normally fall within the range of about 0.01 to about 30 mg/kg of body weight. In the treatment of adult humans, the range of about 0.1 to about 15 mg/kg/day, in single or divided dose, is especially preferred. However, it will be understood that the amount of the compound actually administered will be determined by a physician, in the light of the relevant circumstances, including the condition to be treated, the chosen route of administration, the actual compound or compounds administered, the age, weight, and response of the individual patient, and the severity of the patient's symptoms, and therefore the above dosage ranges are not intended to limit the scope of the invention in any way. In some instances dosage levels below the lower limit of the aforesaid range may be more than adequate, while in other cases still larger doses may be employed without causing any harmful side effect, provided that such larger doses are first divided into several smaller doses for administration throughout the day.

### Method Example 7

### Premenstrual Dysmenorrhoea Dysphoria

Antagonism of vasopressin V₁ₐ receptor has also been shown to alleviate or prevent the symptoms of premenstrual dysmenorrhoea dysphoria (PMDD) and premenstrual dysmenorrhoea CPMD). See generally, Brouard et al, in BJOG 107:614-19 (May 2000). Treatment is illustratively given shortly before the onset of menstruation as a preventative treatment of dysmenorrhoea.

An illustrative assay of vasopressin V₁ₐ antagonists described herein includes a double-blind, randomised, placebo-controlled, cross-over trial in complete block design (such as including three periods and three treatments). Illustrative treatment groups include women ages 18-35 years suffering from primary dysmenorrhoea. Daily dosing is made of either placebo or drug, where the drug dosing is illustratively about 100 mg to about 300 mg of a compound as described herein. The dosing is given in the window from about 4 hours to about three days prior to the onset of bleeding and/or menstrual pain. Alternatively, patients may also be treated with a second daily dose.

Success outcomes include self-reporting of menstrual pain intensity by means of a visual analogue scale, self-rating of symptoms of dysmenorrhoea (including back and pelvic pain) in relation to functional capacity (using a Sultan score), and self-assessment of menstrual blood loss in a menstrual diary record.

### Formulation Example 1

Hard gelatin capsules containing the following ingredients are prepared:

| | Quantity |
|---|---|
| Ingredient | (mg/capsule) |
| Vasopressin antagonist | 30.0 |
| Starch | 305.0 |
| Magnesium stearate | 5.0 |

The above ingredients are mixed and filled into hard gelatin capsules in 340 mg quantities.

### Formulation Example 2

A tablet formula is prepared using the ingredients below:

| | Quantity |
|---|---|
| Ingredient | (mg/tablet) |
| Vasopressin antagonist | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### Formulation Example 3

A dry powder inhaler formulation is prepared containing the following components:

| Ingredient | Weight % |
|---|---|
| Vasopressin antagonist | 5 |
| Lactose | 95 |

The active mixture is mixed with the lactose and the mixture is added to a dry powder inhaling appliance.

### Formulation Example 4

Tablets, each containing 30 mg of active ingredient, are prepared as follows:

| | Quantity | |
|---|---|---|
| Ingredient | (mg/tablet) | |
| Vasopressin antagonist | 30.0 | mg |
| Starch | 45.0 | mg |
| Microcrystalline cellulose | 35.0 | mg |
| Polyvinylpyrrolidone (as 10% solution in water) | 4.0 | mg |
| Sodium carboxymethyl starch | 4.5 | mg |
| Magnesium stearate | 0.5 | mg |
| Talc | 1.0 | mg |
| Total | 120 | mg |

The active ingredient, starch, and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### Formulation Example 5

Capsules, each containing 40 mg of medicament are made as follows:

| | Quantity | |
|---|---|---|
| Ingredient | (mg/capsule) | |
| Vasopressin antagonist | 40.0 | mg |
| Starch | 109.0 | mg |
| Magnesium stearate | 1.0 | mg |
| Total | 150.0 | mg |

The active ingredient, cellulose, starch, and magnesium stearate are blended, passed through a No. 20 mesh U.S. sieve, and filled into hard gelatin capsules in 150 mg quantities.

### Formulation Example 6

Suppositories, each containing 25 mg of active ingredient are made as follows:

| | Quantity |
|---|---|
| Ingredient | (mg) |
| Vasopressin antagonist | 25 mg |
| Saturated fatty acid glycerides to | 2,000 mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation Example 7

Suspensions, each containing 50 mg of medicament per 5.0 ml dose are made as follows:

| | Quantity | |
|---|---|---|
| Ingredient | (mg) | |
| Vasopressin antagonist | 50.0 | mg |
| Xanthan gum | 4.0 | mg |
| Sodium carboxymethyl cellulose(11%) | 50.0 | mg |
| Microcrystalline cellulose (89%) | | |
| Sucrose | 1.75 | g |
| Sodium benzoate | 10.0 | mg |
| Flavor and Color | q.v. | |
| Purified water to | 5.0 | ml |

The medicament, sucrose, and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Formulation Example 8

Capsules, each containing 15 mg of medicament, are made as follows:

| | Quantity |
|---|---|
| Ingredient | (mg/capsule) |
| Vasopressin antagonist | 15.0 mg |
| Starch | 407.0 mg |
| Magnesium stearate | 3.0 mg |
| Total | 425.0 mg |

The active ingredient, cellulose, starch, and magnesium stearate are blended, passed through a No. 20 mesh U.S. sieve, and filled into hard gelatin capsules in 425 mg quantities.

### Formulation Example 9

An intravenous formulation may be prepared as follows:

| | Quantity |
|---|---|
| Ingredient | (mg) |
| Vasopressin antagonist | 250.0 mg |
| Isotonic saline | 1000 ml |

### Formulation Example 10

A topical formulation may be prepared as follows:

| | Quantity | |
|---|---|---|
| Ingredient | (mg) | |
| Vasopressin antagonist | 1-10 | g |
| Emulsifying Wax | 30 | g |
| Liquid Paraffin | 20 | g |
| White Soft Paraffin to | 100 | g |

The white soft paraffin is heated until molten. The liquid paraffin and emulsifying wax are incorporated and stirred until dissolved. The active ingredient is added and stirring is continued until dispersed. The mixture is then cooled until solid.

### Formulation Example 11

Sublingual or buccal tablets, each containing 10 mg of active ingredient, may be prepared as follows:

| | Quantity |
|---|---|
| Ingredient | (mg/tablet) |
| Vasopressin antagonist | 10.0 mg |
| Glycerol | 210.5 mg |
| Water | 143.0 mg |
| Sodium Citrate | 4.5 mg |
| Polyvinyl Alcohol | 26.5 mg |
| Polyvinylpyrrolidone | 15.5 mg |
| Total | 410.0 mg |

The glycerol, water, sodium citrate, polyvinyl alcohol, and polyvinylpyrrolidone are admixed together by continuous stirring and maintaining the temperature at about 90° C. When the polymers have gone into solution, the resulting solution is cooled to about 50-55° C. and the medicament is slowly admixed. The homogenous mixture is poured into forms made of an inert material to produce a drug-containing diffusion matrix having a thickness of about 2-4 mm. This diffusion matrix is then cut to form individual tablets having the appropriate size.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. One such implantable delivery system, used for the transport of biological factors to specific anatomical regions of the body, is described in U.S. Pat. No. 5,011,472, which is herein incorporated by reference.

Indirect techniques, which are generally preferred, usually involve formulating the compositions to provide for drug latentiation by the conversion of hydrophilic drugs into lipid-soluble drugs or prodrugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions that can transiently open the blood-brain barrier.

The type of formulation employed for the administration of the compounds employed in the methods of the present invention may be dictated by the particular compounds employed, the type of pharmacokinetic profile desired from the route of administration and the compound(s), and the state of the patient.

While the invention has been illustrated and described in detail in the foregoing description, such an illustration and description is to be considered as exemplary and not restrictive in character, it being understood that only the illustrative embodiments have been shown and described and that all changes and modifications that come within the spirit of the invention are desired to be protected.

1. A compound of the formula 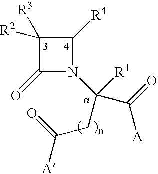
wherein:
n is an integer selected from the group consisting of 0, 1, and 2;
A is R⁵O—, XNH—, or R¹⁴XN—;
A′ is R^{5′}O—, X′NH—, or R^{14′}X′N—;
R¹ is hydrogen or C₁-C₆ alkyl;
R² is C₁-C₆ alkyl, C₂-C₆ alkenyl, C₂-C₆ alkynyl, C₁-C₄ alkoxy, C₁-C₄ alkylthio, halo, haloalkyl, cyano, formyl, alkylcarbonyl, alkoxycarbonyl, or a substituent selected from the group consisting of —CO₂R⁸, —CONR⁸R^{8′}, and —NR⁸(COR⁹);
R³ is a structure selected from the group consisting of 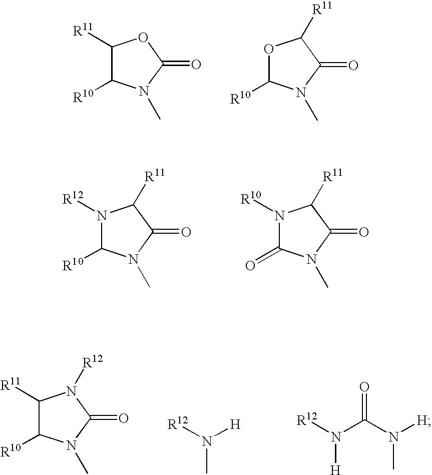
R⁴ is C₁-C₆ alkyl, C₂-C₆ alkenyl, C₂-C₆ alkynyl, C₃-C₈ cycloalkyl, C₃-C₉ cycloalkenyl, C₁-C₃ alkylcarbonyl, optionally substituted aryl, optionally substituted aryl(C₁-C₄ alkyl), optionally substituted aryl(C₂-C₄ alkenyl), or optionally substituted aryl(C₂-C₄ alkynyl);
R⁵ is selected from the group consisting of hydrogen, C₁-C₆ alkyl, C₃-C₈ cycloalkyl, (C₁-C₄ alkoxy)-(C₁-C₄ alkyl), optionally substituted aryl(C₁-C₄ alkyl), Y—, Y—(C₁-C₄ alkyl), and R⁶R⁷N—(C₂-C₄ alkyl);
R^{5′} is selected from the group consisting of hydrogen, C₁-C₆ alkyl, C₃-C₈ cycloalkyl, (C₁-C₄ alkoxy)-(C₁-C₄ alkyl), optionally substituted aryl(C₁-C₄ alkyl), Y′—, Y′—(C₁-C₄ alkyl), and R^{6′}R^{7′}N—(C₂-C₄ alkyl);
Y and Y′ are each independently selected from the group consisting of tetrahydrofuryl, morpholinyl, pyrrolidinyl, piperidinyl, piperazinyl, homopiperazinyl, and quinuclidinyl; where said morpholinyl, pyrrolidinyl, piperidinyl, piperazinyl, homopiperazinyl, or quinuclidinyl is optionally N-substituted with C₁-C₄ alkyl or optionally substituted aryl(C₁-C₄ alkyl);
X is selected from the group consisting of C₁-C₆ alkyl, C₃-C₈ cycloalkyl, (C₁-C₄ alkoxy)-(C₁-C₄ alkyl), optionally substituted aryl, optionally substituted aryl(C₁-C₄ alkyl), optionally substituted aryl(C₃-C₇ cycloalkyl), optionally substituted indan-1-yl, optionally substituted indan-2-yl, optionally substituted 1,2,3,4-tetrahydronaphth-1-yl, optionally substituted 1,2,3,4-tetrahydronaphth-2-yl, Y, Y—(C₁-C₄ alkyl), R⁶R⁷N—, and R⁶R⁷N—(C₂-C₄ alkyl);
R¹⁴ is selected from the group consisting of hydroxy, C₁-C₆ alkyl, C₁-C₄ alkoxycarbonyl, and benzyl; or R¹⁴ and X are taken together with the attached nitrogen atom to form an optionally substituted first heterocycle, where said first heterocycle is selected from the group consisting of pyrrolidinyl, piperidinyl, piperazinyl, homopiperazinyl, pyrrolidinonyl, piperidinonyl, 2-(pyrrolidin-1-ylmethyl)pyrrolidin-1-yl, and 1,2,3,4-tetrahydroisoquinolin-2-yl;
X′ is selected from the group consisting of C₁-C₆ alkyl, C₃-C₈ cycloalkyl, (C₁-C₄ alkoxy)-(C₁-C₄ alkyl), optionally substituted aryl, optionally substituted aryl(C₁-C₄ alkyl), optionally substituted aryl(C₃-C₇ cycloalkyl), optionally substituted indan-1-yl, optionally substituted indan-2-yl, optionally substituted 1,2,3,4-tetrahydronaphth-1-yl, optionally substituted 1,2,3,4-tetrahydronaphth-2-yl, Y′, Y′—(C₁-C₄ alkyl), R^{6′}R^{7′}N—, and R^{6′}R^{7′}N—(C₂-C₄ alkyl);
R^{14′} is selected from the group consisting of hydroxy, C₁-C₆ alkyl, C₁-C₄ alkoxycarbonyl, and benzyl; or R^{14′} and X′ are taken together with the attached nitrogen atom to form an optionally substituted second heterocycle, where said second heterocycle is selected from the group consisting of pyrrolidinyl, piperidinyl, piperazinyl, homopiperazinyl, pyrrolidinonyl, piperidinonyl, 2-(pyrrolidin-1-ylmethyl)pyrrolidin-1-yl, and 1,2,3,4-tetrahydroisoquinolin-2-yl;
R⁶ is hydrogen or C₁-C₆ alkyl; and R⁷ is C₁-C₆ alkyl, C₃-C₈ cycloalkyl, optionally substituted aryl, or optionally substituted aryl(C₁-C₄ alkyl); or R⁶ and R⁷ are taken together with the attached nitrogen atom to form an heterocycle selected from the group consisting of pyrrolidinyl, piperidinyl, morpholinyl, piperazinyl, and homopiperazinyl; where said piperazinyl or homopiperazinyl is optionally N-substitued with R¹³;
R^{6′} is hydrogen or C₁-C₆ alkyl; and R^{7′} is C₁-C₆ alkyl, C₃-C₈ cycloalkyl, optionally substituted aryl, or optionally substituted aryl(C₁-C₄ alkyl); or R^{6′} and R^{7′} are taken together with the attached nitrogen atom to form an heterocycle selected from the group consisting of pyrrolidinyl, piperidinyl, morpholinyl, piperazinyl, and homopiperazinyl; where said piperazinyl or homopiperazinyl is optionally N-substituted with R^{13′};
R⁸ and R^{8′} are each independently selected from the group consisting of hydrogen, C₁-C₆ alkyl, C₃-C₈ cycloalkyl, optionally substituted aryl, and optionally substituted aryl(C₁-C₄ alkyl); or R⁸ and R^{8′} are taken together with the attached nitrogen atom to form an heterocycle selected from the group consisting of optionally substituted pyrrolidinyl, piperidinyl, morpholinyl, piperazinyl, and homopiperazinyl;
R⁹ is selected from the group consisting of hydrogen, C₁-C₆ alkyl, C₃-C₈ cycloalkyl, (C₁-C₄ alkoxy)-(C₁-C₄ alkyl), optionally substituted aryl, optionally substituted aryl(C₁-C₄ alkyl), optionally substituted heteroaryl, optionally substituted heteroaryl(C₁-C₄ alkyl), and R⁸R^{8′}N—(C₁-C₄ alkyl);
R¹⁰ and R¹¹ are each independently selected from the group consisting of hydrogen, optionally substituted C₁-C₆ alkyl, optionally substituted C₃-C₈ cycloalkyl, C₁-C₄ alkoxycarbonyl, C₁-C₅ alkylcarbonyloxy, optionally substituted aryl, optionally substituted aryl(C₁-C₄ alkyl), optionally substituted aryl(C₁-C₄ alkyloxy), optionally substituted aryl(C₁-C₄ alkylcarbonyloxy), diphenylmethoxy, and triphenylmethoxy;
R¹², R¹³, and R^{13′} are each independently selected from the group consisting of hydrogen, C₁-C₆ alkyl, C₃-C₈ cycloalkyl, C₁-C₄ alkoxycarbonyl, optionally substituted aryloxycarbonyl, optionally substituted aryl(C₁-C₄ alkyl), and optionally substituted aryloyl; and
hydrates, solvates, and pharmaceutically acceptable salts thereof.

2. The compound of claim 1, wherein A is XNH—.

3. The compound of claim 1, wherein A is R¹⁴XN—.

4. The compound of claim 3, wherein R¹⁴ is selected from the group consisting of hydroxy, C₁-C₆ alkyl, C₁-C₄ alkoxycarbonyl, and benzyl; and where X is selected from the group consisting of C₁-C₆ alkyl, C₃-C₈ cycloalkyl, (C₁-C₄ alkoxy)-(C₁-C₄ alkyl), optionally substituted aryl, optionally substituted aryl(C₁-C₄ alkyl), optionally substituted aryl(C₃-C₇ cycloalkyl), optionally substituted indan-1-yl, optionally substituted indan-2-yl, optionally substituted 1,2,3,4-tetrahydronaphth-1-yl, optionally substituted 1,2,3,4-tetrahydronaphth-2-yl, Y, Y—(C₁-C₄ alkyl), R⁶R⁷N—, and R⁶R⁷N—(C₂-C₄ alkyl).

5. The compound of claim 3, wherein R¹⁴ and X are taken together with the attached nitrogen atom to form an optionally substituted first heterocycle.

6. The compound of claim 3, wherein R¹⁴ and X are taken together with the attached nitrogen atom to form an optionally substituted first heterocycle substituted with a substituent selected from the group consisting of optionally substituted C₁-C₆ alkyl, optionally substituted C₃-C₈ cycloalkyl, C₁-C₄ alkoxycarbonyl, C₁-C₅ alkylcarbonyloxy, optionally substituted aryl, optionally substituted aryl(C₁-C₄ alkyl), optionally substituted aryl(C₁-C₄ alkyloxy), optionally substituted aryl(C₁-C₄ alkylcarbonyloxy), R⁶R⁷N—, and R⁶R⁷N—(C₁-C₄ alkyl).

7. The compound of claim 3, wherein R¹⁴ and X are taken together with the attached nitrogen atom to form a piperidinyl optionally substituted at the 4-position with hydroxy, C₁-C₆ alkyl, C₃-C₈ cycloalkyl, C₁-C₄ alkoxy, (C₁-C₄ alkoxy)carbonyl, (hydroxy(C₂-C₄ alkyloxy))-(C₂-C₄ alkyl), R⁶R⁷N—, R⁶R⁷N—(C₁-C₄ alkyl), diphenylmethyl, optionally substituted aryl, optionally substituted aryl(C₁-C₄ alkyl), or piperidin-1-yl(C₁-C₄ alkyl).

8. The compound of claim 3, wherein R¹⁴ and X are taken together with the attached nitrogen atom to form a piperazinyl optionally substituted at the 4-position with C₁-C₆ alkyl, C₃-C₈ cycloalkyl, optionally substituted aryl, optionally substituted aryl(C₁-C₄ alkyl), α-methylbenzyl, N—(C₁-C₅ alkyl)acetamid-2-yl, N—(C₃-C₈ cycloalkyl)acetamid-2-yl, R⁶R⁷N—, or (C₁-C₄ alkoxy)carbonyl.

9. The compound of claim 3, wherein R¹⁴ and X are taken together with the attached nitrogen atom to form a homopiperazinyl optionally substituted in the 4-position with C₁-C₄ alkyl, aryl, or aryl(C₁-C₄ alkyl).

10. The compound of claim 1, wherein A′ is XNH—.

11. The compound of claim 1, wherein A′ is R¹⁴XN—.

12. The compound of claim 11, wherein R^{14′} is selected from the group consisting of hydroxy, C₁-C₆ alkyl, C₁-C₄ alkoxycarbonyl, and benzyl; and where X′ is selected from the group consisting of C₁-C₆ alkyl, C₃-C₈ cycloalkyl, (C₁-C₄ alkoxy)-(C₁-C₄ alkyl), optionally substituted aryl, optionally substituted aryl(C₁-C₄ alkyl), optionally substituted aryl(C₃-C₇ cycloalkyl), optionally substituted indan-1-yl, optionally substituted indan-2-yl, optionally substituted 1,2,3,4-tetrahydronaphth-1-yl, optionally substituted 1,2,3,4-tetrahydronaphth-2-yl, Y′, Y′—(C₁-C₄ alkyl), R^{6′}R^{7′}N—, and R^{6′}R^{7′}N—(C₂-C₄ alkyl).

13. The compound of claim 11, wherein R^{14′} and X′ are taken together with the attached nitrogen atom to form an optionally substituted second heterocycle.

14. The compound of claim 11, wherein R^{14′} and X′ are taken together with the attached nitrogen atom to form an optionally substituted second heterocycle substituted with a substituent selected from the group consisting of optionally substituted C₁-C₆ alkyl, optionally substituted C₃-C₈ cycloalkyl, C₁-C₄ alkoxycarbonyl, C₁-C₅ alkylcarbonyloxy, optionally substituted aryl, optionally substituted aryl(C₁-C₄ alkyl), optionally substituted aryl(C₁-C₄ alkyloxy), optionally substituted aryl(C₁-C₄ alkylcarbonyloxy), R^{6′}R^{7′}N—, and R^{6′}R^{7′}N—(C₁-C₄ alkyl).

15. The compound of claim 11, wherein R^{14′} and X′ are taken together with the attached nitrogen atom to form a piperidinyl optionally substituted at the 4-position with hydroxy, C₁-C₆ alkyl, C₃-C₈ cycloalkyl, C₁-C₄ alkoxy, (C₁-C₄ alkoxy)carbonyl, (hydroxy(C₂-C₄ alkyloxy))-(C₂-C₄ alkyl), R^{6′}R^{7′}N—, R^{6′}R^{7′}N—(C₁-C₄ alkyl), diphenylmethyl, optionally substituted aryl, optionally substituted aryl(C₁-C₄ alkyl), or piperidin-1-yl(C₁-C₄ alkyl).

16. The compound of claim 11, wherein R^{14′} and X′ are taken together with the attached nitrogen atom to form a piperazinyl optionally substituted at the 4-position with C₁-C₆ alkyl, C₃-C₈ cycloalkyl, optionally substituted aryl, optionally substituted aryl(C₁-C₄ alkyl), α-methylbenzyl, N—(C₁-C₅ alkyl)acetamid-2-yl, N—(C₃-C₈ cycloalkyl)acetamid-2-yl, R^{6′}R^{7′}N—, or (C₁-C₄ alkoxy)carbonyl.

17. The compound of claim 11, wherein R^{14′} and X′ are taken together with the attached nitrogen atom to form a homopiperazinyl optionally substituted in the 4-position with C₁-C₄ alkyl, aryl, or aryl(C₁-C₄ alkyl).

18. The compound of claim 1, wherein R³ is a structure selected from the group consisting of

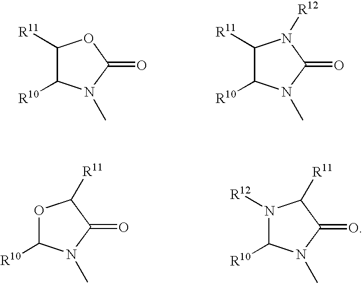

19. The compound of claim 1, wherein R³ is

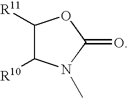

20. The compound of claim 1, wherein R⁴ is optionally substituted aryl(C₁-C₄ alkyl), optionally substituted aryl(C₂-C₄ alkenyl), or optionally substituted aryl(C₂-C₄ alkynyl).

21. The compound of claim 1, wherein R⁴ is optionally substituted aryl(C₂-C₄ alkenyl).

22. The compound of claim 1, wherein R³ is 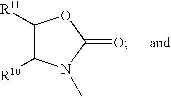
R¹⁰ is optionally substituted phenyl.

23. The compound of claim 18, wherein A is XNH—, where X is optionally substituted aryl(C₁-C₄ alkyl).

24. The compound of claim 18, wherein A′ is R^{14′}X′N—, where R^{14′} and X′ are taken together with the attached nitrogen atom to form an optionally substituted second heterocycle, said optionally second heterocycle selected from the group consisting of piperidinyl and piperazinyl.

25. A pharmaceutical composition comprising the compound of claim 1, where the compound is present in a pharmaceutically effective amount for treating a disease state responsive to antagonism of a vasopressin V₁ₐ receptor in a mammal in need of such treatment; and a pharmaceutically acceptable carrier, diluent, or excipient.

26. (canceled)

27. A method for treating a disease state responsive to antagonism of a vasopressin V₁ₐ receptor in a mammal in need of such treatment, the method comprising the step of administering to the mammal a pharmaceutically effective amount of a composition, said composition comprising the compound of claim 1.

28. The method of claim 27, wherein the composition further comprises a pharmaceutically acceptable carrier, diluent, or excipient.

29. The method of claim 27, wherein the disease state is selected from the group consisting of depression, anxiety, obsessive compulsive disorder, bipolar disorder, primary dysmenorrhoea, and premenstrual dysmenorrhoea dysphoria
